SEQUENCE LISTING (16 sequences)

SEQ ID NO: 1 - protein, 13 aa, Homo sapiens
KEVLLLVHNLPQH

SEQ ID NO: 2 - protein, 14 aa, Homo sapiens
KEVLLLVHNLPQHL

SEQ ID NO: 3 - protein, 15 aa, Homo sapiens
KEVLLLVHNLPQHLF

SEQ ID NO: 4 - protein, 16 aa, Homo sapiens
KEVLLLVHNLPQHLFG

SEQ ID NO: 5 - protein, 15 aa, Homo sapiens
GKEVLLLVHNLPQHL

SEQ ID NO: 6 - protein, 18 aa, Homo sapiens
EGKEVLLLVHNLPQHLFG

SEQ ID NO: 7 - protein, 15 aa, Homo sapiens
YLWWVNGQSLPVSPR

SEQ ID NO: 8 - protein, 25 aa, Homo sapiens
MAISGVPVLGFFIIAVLMSAQESWA

SEQ ID NO: 9 - protein, 5 aa, Homo sapiens
KFERQ

SEQ ID NO: 10 - protein, 15 aa, Homo sapiens
MDDQRDLISNNEQLP

SEQ ID NO: 11 - protein, 4 aa, Homo sapiens
KDEL

SEQ ID NO: 12 - DNA, 2109 bp, Homo sapiens
atggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatag

SEQ ID NO: 13 - protein, 16 aa, Homo sapiens
EGKEVLLLVHNLPQHL

SEQ ID NO: 14 - protein, 17 aa, Homo sapiens
NPPAQYSWLIDGNIQQH

SEQ ID NO: 15 - protein, 18 aa, Homo sapiens
NPPAQYSWLIDGNIQQHT

SEQ ID NO: 16 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

(19) United States
(12) Patent Application Publication
(10) Pub. No.: US 2004/0071726 A1
(43) Pub. Date: Apr. 15, 2004

(54) Peptide epitopes recognized by antigen specific cd4lymphocytes

(76) Inventors: Roman M. Chicz, Belmont, MA (US); Andy Tomlinson, Wayland, MA (US)

Correspondence Address: FISH & RICHARDSON PC, 225 FRANKLIN ST, BOSTON, MA, 02110, US

(21) Appl. No.: 10/380,136

(22) PCT Filed: Sep. 12, 2001
(86) PCT No.: PCT/US01/28467

(51) Int. Cl. G01N 33/53; G01N 33/574; A61K 39/00

(52) U.S. Cl. 424/185.1; 435/7.23

(57) ABSTRACT

The invention provides methods for identifying and validating epitopes that are bound to class II MHC molecules and activate CD4+ T cells involved in the pathogenesis of or protection from diseases, e.g., cancer. The invention includes peptide epitopes (including altered peptide ligands) derived from the CEA polypeptide by such methods, and methods of therapeutic use of these epitopes against diseases such as cancers.

### FIELD OF THE INVENTION

The invention relates to the identification of naturally processed and presented HLA class II restricted peptides and their use as therapeutics and prophylactics.

### BACKGROUND OF THE INVENTION

After internalization and proteolytic processing of intact protein antigens by antigen presenting cells (APCs), class II Major Histocompatibility Complex (MHC) molecules on the APCs bind short antigenic peptides (epitopes) derived from the antigens, presenting the bound peptides to CD4⁺ T lymphocytes [Germain, R. N. (1994), Cell 76:287-299]. Class II MHC genes and the molecules they encode are highly variable among individuals, and differences among the class II MHC molecules determine which peptides are selected for presentation as T cell epitopes. Identification of the naturally processed peptide fragments of a polypeptide of interest that are presented by class II MHC molecules of a subject can be useful for developing peptides that regulate immune response to the polypeptide in that subject.

Carcinoembryonic antigen (CEA) was first described by Gold and Freedman (1965), J. Exp. Med. 121:439-462. CEA is a highly glycosylated, 180,000-dalton protein that is expressed on most gastrointestinal carcinomas, including a large-number of primary and metastatic colorectal tumors. It is also found on some normal, endodermally derived tissues, though in much lower concentrations. CEA cell surface expression can be detected using the monoclonal antibody (mAb) Col-1, and CEA can be identified in total cellular protein by western blot analysis using the same mAb. An MHC class I (HLA-A2)-restricted cytotoxic T lymphocyte (CTL) response against a processed CEA epitope has been described in patients with metastatic disease [Tsang et al. (1995), J. Natl. Cancer. Inst. 87:982-990]. This MHC class I-restricted epitope was identified by taking peripheral blood lymphocytes from patients after immunization with a recombinant vaccinia virus encoding CEA and screening the T cell response against synthetic 9- to 11-mer CEA peptides. CTLs from three patients were shown to have specificity for a class I-restricted CEA epitope in an assay using autologous Epstein Barr Virus (EBV)-transformed B cells pulsed with a 9 amino acid synthetic peptide derived from CEA.

Generally, HLA class II-restricted epitopes are different from class I-restricted epitopes, and produce different immunological responses. Epitopes presented by HLA class II molecules are generally, although not exclusively, recognized by CD4⁺ T cells. The HLA class II restricted CD4⁺ T cell response can act as a helper response, providing help to HLA class I-restricted CTL. Class II-restricted responses may also result in the recruitment of cells such as tumoricidal macrophages and eosinophils that work in unison to eliminate neoplastic cells. Furthermore, recent evidence suggests that T helper cells can elicit an anti-angiogenic response in the microenvironment of a tumor mass, thereby cutting off the blood supply to the rapidly dividing cells.

The potential role of CD4⁺ T cells in antitumor immunity and cancer immunotherapy has been the subject of general review [Topalian (1994), Current Opinion in Immunology 6:741-745; Pardoll and Topalian (1998), Current Opinion in Immunology 10:588-594; Toe et al. (1999), J. Exp. Med. 189:753-756]. Among other functions, CD4⁺ T cells provide help and immunological memory, can play an important role in initiating and maintaining specific anti-tumor immunity, and may be instrumental in the phenomenon of tumor-specific T cell tolerance.

### SUMMARY OF THE INVENTION

The invention features methods for identifying peptide epitopes that activate CD4⁺ T lymphocyte responses involved in the initiation, promotion, or exacerbation of certain diseases.

The invention is based on the discovery that one can identify HLA class II-restricted epitopes naturally produced by APC transfected with DNA encoding a protein from which the epitopes are derived. These peptide epitopes can be incorporated into a drug delivery system and used to elicit an epitope-specific CD4⁺ T cell response in an appropriate mammalian subject. Where the epitope is derived from a tumor antigen, such a response promotes the migration of CD8+CTL to the vicinity of tumor cells expressing the tumor antigen and specific targeting of the tumor cells by the CTL. This CD4⁺ T lymphocyte response could also promote migration of tumoricidal macrophages and eosinophils to the vicinity of the tumor cells to help kill the tumor cells. Finally, the CD4⁺ T cell response can also produce an anti-angiogenic effect at the microenvironment of the tumor, cutting off the nutrient supply needed to continue cell division and tumor growth.

Altered peptide ligands (APL), which are class II MHC-binding variant peptides in which 1 to 6 amino acid residues are different from the corresponding residues of the wild-type class II MHC-binding peptide, but which still bind to the same class II MHC molecules as the wild-type peptides, are also encompassed by the invention, as are methods of therapy and prophylaxis involving the use of APL. APL have the ability to elicit different patterns of cytokine production in CD4⁺ T cells than do their parent wild-type peptides. Thus, for example, while a wild-type peptide presented by a class II MHC molecule may induce production of Th1 cytokines, an APL derived from it and presented by the same class II MHC molecule may elicit Th2 or other immunoregulatory cytokines. Alternatively, the wild-type peptide may stimulate the production of Th2 cytokines while a corresponding APL elicits production of Th1 cytokines.

More specifically, the invention features a method of identifying a class II MHC-binding fragment of a polypeptide. The method involves: (a) providing a mammalian antigen-presenting cell (APC) cell line transfected with a DNA encoding a polypeptide; (b) isolating from the APC a class II MHC molecule bound to a peptide, the peptide being a class II MHC-binding fragment of the polypeptide; (c) eluting the peptide from the class II MHC molecule; and (d) identifying the peptide as a fragment of the polypeptide. The polypeptide can have the sequence of a tumor antigen (e.g., CEA). The APC can be any class II MHC-expressing mammalian cell, e.g., a dendritic cell, a macrophage, a monocyte or a B lymphocyte, and the mammal from which it is derived can be a human. The class II MHC molecule can be a HLA-DR molecule, or a HLA-DQ molecule, or a HLA-DP molecule. Such a DR molecule can have a β-chain encoded by a DRB1*0401, DRB1*0101, DRB1*0301, DRB1*0701, DRB1*1101, DRB1*1301, DRB1*1302, DRB1*1501, or DRB5*0101 gene.

The invention provides an isolated peptide fewer than 35 (e.g., fewer than 33, 30, 26, 22, 19, 18, 17, 16, 15, or 14) amino acid residues in length and including the sequence KEVLLLVHNLPQH (SEQ ID NO:1). The peptide thus can include or be the amino acid sequence KEVLLLVHNLPQHL (SEQ ID NO:2); KEVLLLVHNLPQHLF (SEQ ID NO:3); KEVLLLVHNLPQHLFG (SEQ ID NO:4); GKEVLLLVHNLPQHL (SEQ ID NO:5); EGKEVLLLVHNLPQHLFG (SEQ ID NO:6); or EGKEVLLLVHNLPQHL (SEQ ID NO:13). Also embraced by the invention is a peptide fewer than 35 (e.g., fewer than 33, 30, 26, 22, 19, 18, 17, 16, 15, or 14) amino acid residues in length that includes or is the sequence YLWWVNGQSLPVSPR (SEQ ID NO:7), optionally with additional CEA sequence on one or both ends. The invention also includes a peptide fewer than 35 (e.g., fewer than 33, 30, 26, 22, 19, 18, 17, 16, 15, or 14) amino acid residues in length and including the sequence NPPAQYSWLIDGNIQQH (SEQ ID NO:14). The peptide thus can include or be the amino acid sequence NPPAQYSWLIDGNIQQH (SEQ ID NO:14) or NPPAQYSWLIDGNIQQHT (SEQ ID NO:15).

Another embodiment of the invention is an altered peptide ligand (APL) the amino acid sequence of which is identical, except for 1-6 amino acid substitutions, to a fragment of carcinoembryonic antigen (CEA), the fragment being fewer than 35 (e.g., fewer than 33, 30, 26, 22, 19, 18, 17, 16, 15, or 14) amino acids residues in length and including or being the sequence KEVLLLVHNLPQH (SEQ ID NO:1), YLWWVNGQSLPVSPR (SEQ ID NO:7), or NPPAQYSWLIDGNIQQH(SEQ ID NO:14), optionally with additional CEA sequence on one or both ends. No more than 30% of the amino acid residues of the fragment are substituted with different amino acid residues in the APL, and the APL can bind to a class II MHC molecule. The sequence of the fragment can be, for example, KEVLLLVHNLPQH (SEQ ID NO:1), YLWWVNGQSLPVSPR (SEQ ID NO:7), or NPPAQYSWLIDGNIQQH (SEQ ID NO:14), optionally with additional CEA sequence on one or both ends. In this method, the mammal can be one suspected of having or being susceptible to cancer.

The invention also features a process for making an APL. The method involves: (a) carrying out the above method of identifying a class II MHC-binding fragment of a polypeptide, and (b) synthesizing an APL consisting of a sequence which is identical to that of the peptide, except having amino acid substitutions at 1, 2, 3, 4, 5, or 6 positions in the peptide. The polypeptide can be a tumor antigen such as CEA.

Another aspect of the invention is a method of activating T cell reactivity in a mammal. The method involves: (a) providing (i) a peptide, the sequence of which consists of the sequence of a naturally processed fragment of CEA, the peptide being capable of binding to a class II MHC molecule of the mammal and of eliciting a CD4⁺ T cell response, or (ii) a DNA encoding a polypeptide that is (1) the peptide, (2) the peptide plus an amino terminal methionine residue, and (3) either (1) or (2) linked to a trafficking sequence; and (b) administering the peptide or DNA to the mammal. The peptide can include or be KEVLLLVHNLPQH (SEQ ID NO:1), KEVLLLVHNLPQHL (SEQ ID NO:2), KEVLLLVHNLPQHLF (SEQ ID NO:3), KEVLLLVHNLPQHLFG (SEQ ID NO:4), GKEVLLLVHNLPQHL (SEQ ID NO:5), EGKEVLLLVHNLPQHLFG (SEQ ID NO:6), YLWWVNGQSLPVSPR (SEQ ID NO:7), EGKEVLLLVHNLPQHL (SEQ ID NO:13), NPPAQYSWLIDGNIQQH (SEQ ID NO:14), or NPPAQYSWLIDGNIQQHT (SEQ ID NO:15), optionally with additional CEA sequence on one or both ends.

The invention also provides a method of altering a T cell response in a mammal. The method involves: (a) providing (i) an APL having a sequence identical, except for amino acid substitutions at 1-6 positions, to the sequence of a naturally-processed fragment of CEA, the APL being able to bind to a class II MHC molecule of the mammal, or (ii) a DNA encoding a polypeptide that can be (1) the APL, (2) the APL plus an amino terminal methionine residue, and (3) either (1) or (2) linked to a trafficking sequence; and (b) administering the APL or DNA to the mammal.

The above described method of identifying a class II MHC-binding fragment of a polypeptide can include the additional steps of: (e) providing CD4⁺ lymphocytes from a mammal having a condition suspected of being associated with presentation of the peptide by the class II MHC molecule, the APCs of the mammal bearing the class II MHC molecule; (f) providing a population of APCs that bear the class II MHC molecule with the peptide bound thereto; (g) contacting the population of APCs of (f) with the CD4⁺ lymphocytes of (e); and (h) determining whether the CD4⁺ lymphocytes recognize the class II MHC-bound peptide, as an indication that presentation of the peptide to CD4⁺ T lymphocytes is associated with the condition. The presentation can be associated with a pathological response of CD4⁺ T lymphocytes or a protective response of CD4⁺ T lymphocytes.

Another aspect of the invention is a method of diagnosis that involves: (a) providing a CD4⁺ lymphocyte from an individual suspected of having or being susceptible to cancer; (b) providing an APC which bears on its surface a class II MHC molecule of an allele identical to one expressed by the individual, the class II MHC molecule being bound to a CEA peptide; (c) contacting the APC with the CD4⁺ lymphocyte; and (d) determining whether the CD4⁺ lymphocyte recognizes the class II MHC-bound peptide, as an indication that the individual has or is susceptible to cancer. The peptide can include or be the amino acid sequence KEVLLLVHNLPQH (SEQ ID NO:1); KEVLLLVHNLPQHL (SEQ ID NO:2;) KEVLLLVHNLPQHLF (SEQ ID NO:3); KEVLLLVHNLPQHLFG (SEQ ID NO:4); GKEVLLLVHNLPQHL (SEQ ID NO:5); EGKEVLLLVHNLPQHLFG (SEQ ID NO:6); YLWWVNGQSLPVSPR (SEQ ID NO:7); EGKEVLLLVHNLPQHL (SEQ ID NO:13); NPPAQYSWLIDGNIQQH (SEQ ID NO:14); or NPPAQYSWLIDGNIQQHT (SEQ ID NO:15), optionally with additional CEA sequence on one or both ends.

The invention also features a method of treating a subject suspected of having or being susceptible to cancer. The method involves administering a peptide to the subject. The peptide can be fewer than 35 (e.g., fewer than 33, 30, 26, 22, 19, 18, 17, 16, 15, or 14) amino acid residues in length and include a sequence KEVLLLVHNLPQH (SEQ ID NO:1), optionally with additional CEA sequence on one or both ends. Alternatively, the peptide can be fewer than 35 (e.g., fewer than 33, 30, 26, 22, 19, 18, 17, 16, 15, or 14) amino acid residues in length and include or be the sequence YLWWVNGQSLPVSPR (SEQ ID NO:7), optionally with additional CEA sequence on one or both ends. Furthermore, the peptide can be fewer than 35 (e.g., fewer than 33, 30, 26, 22, 19, 18, 17, 16, 15, or 14) amino acid residues in length and include or be the sequence NPPAQYSWLIDGNIQQH (SEQ ID NO:14), optionally with additional CEA sequence on one or both ends.

The invention also provides a method of identifying a reagent for diagnosing cancer. The method involves: (a) providing a test reagent that can be a Fab fragment, a monoclonal antibody (mAb), or a single chain Fv (scFv) fragment; (b) providing a complex that contains a class II MHC molecule bound to a peptide that includes or is KEVLLLVHNLPQH (SEQ ID NO:1); KEVLLLVHNLPQHL (SEQ ID NO:2); KEVLLLVHNLPQHLF (SEQ ID NO:3); KEVLLLVHNLPQHLFG (SEQ ID NO:4); GKEVLLLVHNLPQHL (SEQ ID NO:5); EGKEVLLLVHNLPQHLFG (SEQ ID NO:6); YLWWVNGQSLPVSPR (SEQ ID NO:7); EGKEVLLLVHNLPQHL (SEQ ID NO:13); NPPAQYSWLIDGNIQQH (SEQ ID NO:14); or NPPAQYSWLIDGNIQQHT (SEQ ID NO:15); and (c) testing whether the test reagent binds to the complex. The class II MHC molecule can be a DR molecule with a β-chain encoded by a DRB1*0401 gene or by a DRB1*0101 gene. As used herein, a “scFv” fragment is a recombinant fragment of an antibody molecule that contains, in a single polypeptide chain, the antigen-binding regions of an immunoglobulin (Ig) heavy and an Ig light chain. scFv fragments generally either contain (a) no Ig heavy or Ig light chain constant regions or (b) less than the whole constant region of an Ig heavy and/or Ig light chain.

Also embraced by the invention is a method of diagnosis. The method involves:(a) providing a test cell from a mammalian subject; (b) providing a reagent that binds to a CEA peptide fragment bound to a class II MHC molecule; (c) contacting the test cell with the reagent; and (d) detecting binding of the reagent to the test cell as an indication that the test cell is a cancer cell.

The invention also features a method of cancer therapy that involves: (a) providing a composition that includes a reagent that can be a Fab fragment, a mAb, or a scFv fragment, the reagent being able to recognize a naturally processed CEA peptide bound to a MHC class II molecule and being linked to an agent that can be a chemotherapeutic compound, a radioactive isotope or a toxin; and (b) administering the composition to a subject suspected of having or being susceptible to a cancer characterized by expression of CEA.

Another aspect of the invention is a method of identifying a class II MHC-binding fragment of a tumor antigen. The method involves: (a) providing a mammalian APC that contains a class II MHC molecule and the tumor antigen; (b) isolating from the APC the class II MHC molecule bound to a peptide, the peptide being a class II MHC binding fragment of the tumor antigen; (c) eluting the peptide from the class II MHC molecule; and (d) identifying the amino acid sequence of the peptide. The method can involve the additional steps of: (e) providing CD4⁺ lymphocytes from a mammal having a cancer suspected of being associated with presentation of the peptide by the class II MHC molecule, the APCs of the mammal bearing the class II MHC molecule; (f) providing a population of APCs that bear the class II MHC molecule with the peptide bound thereto; (g) contacting the population of APCs of (f) with the CD4⁺ lymphocytes of (e); and (h) determining whether the CD4⁺ lymphocytes recognize the class II MHC bound peptide, as an indication that presentation of the peptide to CD4⁺ lymphocytes is associated with the cancer.

The invention also provides an isolated DNA that contains a nucleotide sequence that encodes a peptide fewer than 35 (e.g., fewer than 33, 30, 26, 22, 19, 18, 17, 16, 15, or 14) amino acid residues in length, and that contains the sequence KEVLLLVHNLPQH (SEQ ID NO:1). The peptide encoded by the DNA thus can include or be, for example, the amino acid sequence KEVLLLVHNLPQHL (SEQ ID NO:2); KEVLLLVHNLPQHLF (SEQ ID NO:3); KEVLLLVHNLPQHLFG (SEQ ID NO:4); GKEVLLLVHNLPQHL (SEQ ID NO:5); EGKEVLLLVHNLPQHLFG (SEQ ID NO:6); or EGKEVLLLVHNLPQHL (SEQ ID NO:13), optionally with additional CEA (or non-CEA) sequence on one or both ends. Also embraced by the invention is DNA encoding a peptide fewer than 35 (e.g., fewer than 33, 30, 26, 22, 19, 18, 17, 16, 15, or 14) amino acid residues in length that includes or is the sequence YLWWVNGQSLPVSPR (SEQ ID NO:7), optionally with additional CEA sequence on one or both ends. The invention also features an isolated DNA that contains a nucleotide sequence that encodes a peptide fewer than 35 (e.g., fewer than 33, 30, 26, 22, 19, 18, 17, 16, 15, or 14) amino acid residues in length, and that contains the sequence NPPAQYSWLIDGNIQQH (SEQ ID NO:14). The peptide encoded by the DNA thus can include or be, for example, the amino acid sequence NPPAQYSWLIDGNIQQH (SEQ ID NO:14), or NPPAQYSWLIDGNIQQHT (SEQ ID NO:15), optionally with additional CEA sequence on one or both ends. Also featured is a vector containing the above DNA of the invention. In the vector the nucleotide sequence can be operatively linked to a transcriptional regulatory element. Also included in the invention is a cell (e.g., a mammalian, an insect, a bacterial, a yeast, or a fungal cell) containing any of the vectors of the invention.

Another aspect of the invention is an isolated peptide containing a fragment of CEA that includes: (a) KEVLLLVHNLPQH (SEQ ID NO:1) and a further 1-15 residues of CEA sequence at the C-terminus of SEQ ID NO:1, at the N-terminus of SEQ ID NO:1, or at both the N-terminus and the C-terminus of SEQ ID NO:1; (b) YLWWVNGQSLPVSPR (SEQ ID NO:7) and a further 1-15 residues of CEA sequence at the C-terminus of SEQ ID NO:7, at the N-terminus of SEQ ID NO:7, or at both the N-terminus and the C-terminus of SEQ ID NO:7; or (c) NPPAQYSWLIDGNIQQH (SEQ ID NO:14) and a further 1-15 residues of CEA sequence at the C-terminus of SEQ ID NO:14, at the N-terminus of SEQ ID NO:14, or at both the N-terminus and the C-terminus of SEQ ID NO:14. This peptide can be used in a method of activating T cell reactivity in a mammal. The method involves: (a) providing (i) any of the peptide or (ii) a DNA encoding a polypeptide which can be (1) the peptide, (2) the peptide plus an amino terminal methionine residue, or (3) either (1) or (2) linked to a trafficking sequence; and (b) administering the peptide or DNA to the mammal.

Another embodiment of the invention is an isolated fragment of CEA shorter than full-length CEA; the fragment includes one or more amino acid sequences selected from the group consisting of SEQ ID NO:1, SEQ ID NO:7, and SEQ ID NO:14. This fragment of CEA can be used in a method of activating T cell responsiveness in a mammal. The method involves: (a) providing (i) the CEA fragment, or (ii) a DNA encoding a polypeptide that can be (1) the CEA fragment, (2) the CEA fragment plus an amino terminal methionine residue, and (3) either (1) or (2) linked to a trafficking sequence, the DNA not encoding full-length CEA; and (b) administering the fragment or DNA to the mammal.

Another method of activating T cell responsiveness in a mammal involves: (a) administering to the mammal (i) CEA or (ii) a DNA encoding CEA; and (b) testing CD4⁺ T cells of the animal for responsiveness to a naturally processed fragment of CEA (e.g., fragments with SEQ ID NOS:1-7 and 13-15).

The invention also provides an isolated peptide that includes: (a) at least one CEA segment that can be a segment with SEQ ID NO:1, SEQ ID NO:7, and SEQ ID NO:14; and (b) one or more amino acids at the C-terminus of the CEA segment, at the N-terminus of the CEA segment, or at both the C-terminus and the N-terminus of the CEA segment, the peptide having an amino acid sequence that is not identical to that of full-length CEA. This peptide can be used in a method of activating T cell reactivity in a mammal. The method involves: (a) providing (i) the peptide, or (ii) a DNA encoding a polypeptide that can be (1) the peptide, (2) the peptide plus an amino terminal methionine residue, and (3) either (1) or (2) linked to a trafficking sequence, the DNA not encoding full-length CEA; and (b) administering the peptide or DNA to the mammal.

An “isolated” peptide of the invention is a peptide that either has no naturally-occurring counterpart (e.g., an APL), or has been separated or purified from components which naturally accompany it, e.g., in tissues such as pancreas, liver, spleen, ovary, testis, muscle, joint tissue, neural tissue, gastrointestinal tissue, or body fluids such as blood, serum, or urine. Such a naturally-occurring peptide is considered “isolated” when it is at least 70%, by dry weight, free from the proteins and peptides with which it is naturally associated. Preferably, a preparation of a peptide of the invention is at least 80%, more preferably at least 90%, and most preferably at least 99%, by dry weight, the peptide of the invention. Since a peptide that is chemically synthesized is, by its nature, separated from the components that naturally accompany it in the tissue where it occurs naturally, the synthetic peptide is, by definition, “isolated.”

An isolated peptide of the invention can be obtained, for example, by extraction from a natural source (e.g., from human tissues or bodily fluids); by expression of a recombinant nucleic acid encoding the peptide; or by chemical synthesis. A peptide that is produced in a cellular system different from the source from which it naturally originates is “isolated,” because it will be separated from components which naturally accompany it. The extent of isolation or purity can be measured by any appropriate method, e.g., column chromatography, polyacrylamide gel electrophoresis, mass analysis, or HPLC analysis.

An “isolated DNA” means DNA free of the genes that flank the gene of interest (e.g., the gene encoding CEA) in the genome of the organism in which the gene of interest naturally occurs. The term therefore includes a recombinant DNA incorporated into a vector, into an autonomously replicating plasmid or virus, or into the genomic DNA of a prokaryote or eukaryote. It also includes a separate molecule such as: a cDNA where the corresponding genomic DNA has introns and therefore a different sequence; a genomic fragment; a fragment produced by polymerase chain reaction (PCR); a restriction fragment; a DNA encoding a non-naturally occurring protein, fusion protein, or fragment of a given protein; or a nucleic acid which is a degenerate variant of a naturally occurring nucleic acid. In addition, it includes a recombinant nucleotide sequence that is part of a hybrid gene, i.e., a gene encoding a fusion protein. Also included is a recombinant DNA that includes a DNA sequence that encodes any of the peptides with SEQ ID NOS:1-7 and 13-15.

As used herein, “protection from a mammalian disease” means prevention of onset of a mammalian disease or lessening the severity of a disease existing in a mammal. “Prevention” can include a delay of onset, as well as a partial or complete block in progress of the disease or disease symptoms.

As used herein, “a naturally-processed peptide fragment” is a peptide fragment produced by proteolytic degradation of a protein in an antigen presenting cell of a mammal. As used herein, a “tumor antigen” is a molecule (e.g., a protein molecule) that is expressed by a tumor cell. Such a molecule can differ (e.g., by one or more amino acid residues where the molecule is a protein) from, or it can be identical to, its counterpart expressed in normal cells. It is preferably not expressed by normal cells. Alternatively, it is expressed at a higher level (e.g., a two-fold, three-fold, five-fold, ten-fold, 20-fold, 40-fold, 100-fold, 500-fold, 1,000-fold, 5,000-fold, or 15,000-fold higher level) in a tumor cell than in the tumor cell's normal counterpart. Examples of tumor antigens include, without limitation, CEA, prostate specific antigen (PSA), MAGE (melanoma antigen) 1-4, 6 and 12, MUC (mucin) (e.g., MUC-1, MUC-2, etc.), tyrosinase, MART (melanoma antigen), Pmel 17(gp100), GnT-V intron V sequence (N-acetylglucoaminyltransferase V intron V sequence), Prostate Ca psm, PRAME (melanoma antigen), β-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67. Recognition of such a peptide by CD4⁺ T cells of a mammal (e.g., a human patient) is indicative of the existence, or future onset, of cancer in the mammal.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. Unless otherwise indicated, these materials and methods are illustrative only and are not intended to be limiting. All publications, patent applications, patents and other references mentioned herein are incorporated by reference.

Other features and advantages of the invention, e.g., methods of identifying peptides that activate CD4⁺ T lymphocyte responses, will be apparent from the following description, from the drawings and from the claims.

### BRIEF DESCRIPTION OF THE DRAWINGS

FIG. 1A is a depiction of the nucleotide sequence of CEA-encoding cDNA (SEQ ID NO:12) that was inserted into the expression vector pcDNA3.1-Zeo.

FIG. 1B is a depiction of the amino acid sequence of CEA (SEQ ID NO:17) encoded by the cDNA that was inserted into the expression vector pcDNA3.1-Zeo.

FIG. 2 is a histogram showing the fluorescence-activated flow cytometric profiles of JY cells that were stained with CEA specific murine mAb and fluorescein isothiocyanate (FITC)-conjugated goat antibody specific for murine immunoglobulin.

FIG. 3 is a diagram showing two appropriately aligned matrix assisted laser desorption time-of-flight (MALDI-TOF) mass spectometry spectra derived from two mixtures of peptides in which separate aliquots of peptide mixtures prepared from A2.140-pcDNA3 and A2.140-CEA-38 cells were analyzed by IMF procedures.

FIG. 4 is a MS/MS Spectrum of naturally processed KEVLLLVHNLPQH (SEQ ID NO: 1). The two background responses were derived from a peptide that co-eluted chromatographically with KEVLLLVHNLPQH (SEQ ID NO: 1) and was closely related in M/Z. These peptides (KEVLLLVHNLPQH (SEQ ID NO: 1) and the co-eluting species) could be only partially resolved chromatographically, but this did not prevent the identification of KEVLLLVHNLPQH (SEQ ID NO: 1) from the resultant composite mass spectrum. Spectral labeling in FIGS. 3-9 use the nomenclature described by Roepstorff and Fohlman (1988), Biomed. Mass. Spectrom. 11:601, and Biemann (1990), Meth. Enzymol. 193:866-867.

FIG. 5 is a MS/MS spectrum of naturally processed KEVLLLVHNLPQHL (SEQ ID NO:2)

FIG. 6 is a MS/MS spectrum of naturally processed KEVLLLVHNLPQHLF (SEQ ID NO:3).

FIG. 7 is a MS/MS spectrum of naturally processed KEVLLLVHNLPQHLFG (SEQ ID NO:4).

FIG. 8 is a MS/MS spectrum of naturally processed GKEVLLLVHNLPQHL (SEQ ID NO:5).

FIG. 9 is a MS/MS spectrum of naturally processed EGKEVLLLVHNLPQHLFG (SEQ ID NO:6).

FIG. 10 is a MS/MS spectrum of naturally processed YLWWVNGQSLPVSPR (SEQ ID NO:7).

FIG. 11 is a MS/MS spectrum of naturally processed EGKEVLLLVHNLPQHL (SEQ ID NO:13).

FIG. 12 is a MS/MS spectrum of naturally processed NPPAQYSWLIDGNIQQH (SEQ ID NO:14).

FIG. 13 is a MS/MS spectrum of naturally processed NPPAQYSWLIDGNIQQHT (SEQ ID NO:15).

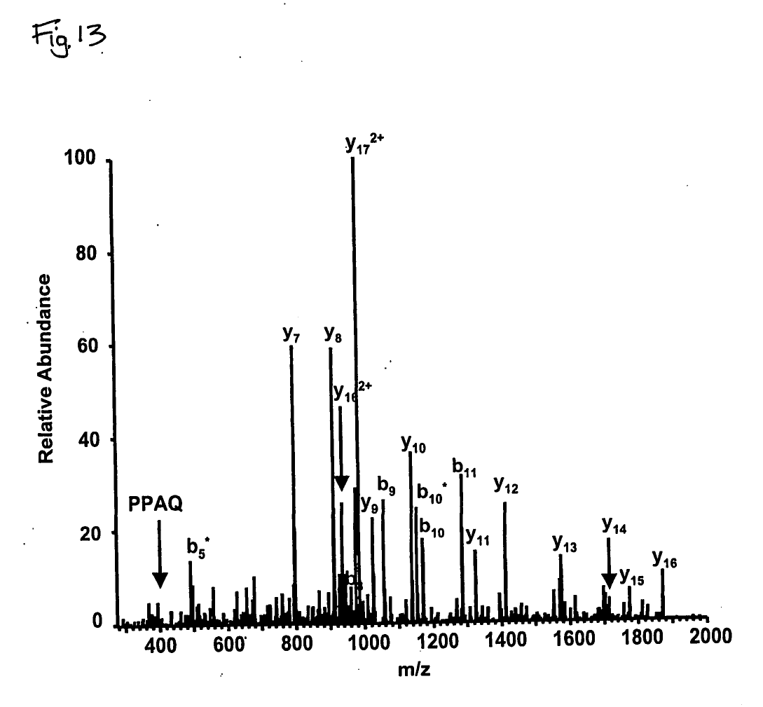

### DETAILED DESCRIPTION

The present invention is based on the novel discovery that, by transfecting an APC with an expression vector containing a DNA sequence encoding a polypeptide of interest (PPI), the APC will synthesize the PPI and then transport it to one of the antigen-processing organelles within the cell, e.g., endosomes, lysozomes and structures designated “MHC class II compartments” (MIIC) that have lysosomal characteristics and are enriched for class II MHC molecules but are substantially devoid of class I MHC molecules [Peter et al. (1991), Nature 349:669-676]. The PPI is degraded by proteolytic enzymes into peptide fragments. If any of these peptide fragments has, by virtue of its length and sequence, the ability to bind to one of the class II MHC molecules expressed by the APC, it will do so in the antigen processing organelle. The resulting peptide-class II MHC molecular complex is then transported to the APC's cell membrane, where it becomes available for interaction with CD4⁺ T cells bearing antigen-specific receptors that specifically recognize that particular peptide-class II MHC complex. By eluting peptides from class II MHC molecules isolated from these APC, a set of naturally processed peptides derived from the PPI, as well as from other polypeptides of intracellular or extracellular origin, is obtained. The peptides, which are specific to the particular types (isotypes and alleles) of class II MHC molecules expressed by the APC, are then chemically separated and their amino acid sequences determined. By comparison of the peptide amino acid sequences to the sequence of the PPI, it is possible to identify those which are derived from the PPI. Thus, the present invention includes a method of identifying peptide fragments that are naturally processed by APC and have intrinsic binding affinity for the relevant class II MHC molecule. The method can be invaluable for identifying peptides derived from a polypeptide suspected of being an antigen that activates CD4⁺ T cells involved in either (a) the pathogenesis (pathology) of a disease, especially one in which susceptibility or protection is known to be associated with expression of a particular type of class II MHC molecule, or (b) prevention or reduction of the symptoms of a disease, especially one in which protection or a reduction in severity is associated with expression of a particular type of class II MHC molecule. The method is designated “Immunological Mass Fingerprinting” (“IMF”).

The described method ensures that the peptides identified are those that both (i) are naturally processed in vivo by the APC, and (ii) become associated, in the APC, with the relevant class II MHC molecules.

Furthermore, the present method controls for class II MHC type, an important aspect essential to link any given peptide to a particular CD4⁺ T cell-mediated disease in a given individual but especially important in disorders in which class II MHC type determines disease susceptibility or resistance.

Any naturally processed peptide with a sequence that corresponds to a fragment of the PPI, and which binds to a class II MHC molecule associated with the disease of interest, could be a peptide that activates CD4⁺ T cells that either initiate, promote, or exacerbate the disease or mediate immunity to it. To obtain confirmatory evidence of this possibility, test CD4⁺ T cells from subjects expressing the relevant class II MHC molecules can be assayed for responsiveness to a peptide identified in accordance with the invention. Control CD4⁺ T cells can be from subjects also expressing the class II MHC molecule but without symptoms of the disease. A significant response of the test CD4⁺ T cells and no or little response of the control CD4⁺ T cells would indicate that the relevant peptide is involved either in the disease process (pathology of the disease) or in immunity to the disease. The cellular response phase of the method is designated “Epitope Verification” (“EV”).

By applying the methods of the invention to the tumor antigen CEA, CEA-derived peptides were identified as epitopes that could be involved in the pathogenesis of cancer in human patients expressing the DR4 or DR1 class II MHC allele. Based on their amino acid sequences, these peptides fall into 3 nested groups. A consensus peptide corresponding to the core region of each nested group can be synthesized and tested for its ability to activate CD4⁺ T cells from either DR4, DR13, or DR1-expressing cancer patients or DR4, DR13, or DR1-expressing subjects without disease symptoms.

The methods of the invention can be applied to identifying peptides involved in the pathogenesis of or protection from any of a wide range of diseases, especially those in which relative susceptibility or resistance has been associated with expression of a particular class II MHC allele, provided that the amino acid sequence (or partial amino acid sequence) of a suspect polypeptide antigen is available. Candidate diseases include, without limitation, infectious diseases (e.g., diseases caused by Chlamydia trachomatis, Helicobacter pylori, Neisseria meningitidis, Mycobacterium leprae, M. tuberculosis, Measles virus, hepatitis C virus, human immunodeficiency virus, and Plasmodium falciparium), cancer (e.g. melanoma, ovarian cancer, breast cancer, colon cancer and B cell lymphomas) [Topalian (1994), Curr. Opinion in Immunol. 6: 741-745; Topalian et al. (1996), J. Exp. Med. 183: 1965-1971], and autoimmune diseases (e.g., insulin dependent diabetes mellitus, rheumatoid arthritis, multiple sclerosis, myasthenia gravis, and systemic lupus erythmatosus).

The invention also includes peptides derived from CEA using the above method, as described in Example 1. Also included in the invention are APL derived from the CEA peptides by replacing 1 to 6 (i.e., 1, 2, 3, 4, 5, or 6) amino acid residues of a naturally processed peptide that activates an immune response. Each residue is replaced with a different residue, resulting in a variant peptide that still binds to the same class II MHC allele, but elicits qualitatively different responses in CD4⁺ T cells than does the parent peptide from which the APL is derived. Thus, APL have the potential to be therapeutic and/or prophylactic in diseases in which the CD4⁺ T cell response to the relevant parent peptide is pathogenic. The invention features methods of therapy and prophylaxis involving the use of APL. The invention also features use of disease-related peptides in the diagnosis of disease or monitoring immune-based therapy.

1. Methods of Identifying CD4⁺ T Cell Activating Peptide Epitopes Derived from Polypeptide Antigens

The methods of the invention have two distinct phases. The first is termed “Immunological Mass Fingerprinting” (IMF) and the second “Epitope Verification” (EV). The purpose of the IMF is to direct a candidate polypeptide to any one of the antigen processing compartments of an APC where it can be degraded to peptide fragments. Any peptides of the appropriate length (about 9 to 25 amino acid residues), and having specific binding affinity for a particular class II MHC molecule expressed by the APC, will bind to that class II MHC molecule in the antigen processing compartments. At least some of these peptide-class II MHC molecular complexes then migrate to the cell membrane of the APC. The complexes (both cell-membrane associated and intracellular) are isolated from the APC and the peptides eluted from the complexes. The eluted peptides are then separated, their amino acid sequences determined, and the sequences compared to that of the candidate polypeptide.

The IMF can generally be applied to the analysis of peptides produced by an APC expressing defined class II MHC molecules. As such, the method can be useful for basic research studies, e.g., studies aimed at identifying amino acid residues in a polypeptide that determine sites of “cutting” by the proteolytic antigen processing enzymes of APC. Alternatively, where the polypeptide is suspected of being an antigen that activates CD4⁺ T cells that cause or promote a particular disease or mediate protection from a disease, the IMF can be used to identify disease-related or protective peptide epitopes derived from the polypeptide. This information would be useful for basic research into the etiology of the disease, or as a basis for development of diagnostics, therapeutics, or vaccines for the disease.

A peptide whose amino acid sequence matches that of a region of the candidate polypeptide is likely to be one that activates CD4⁺ T cells involved in the pathogenesis of or immunity to the relevant disease. Such a peptide can be subjected to the EV procedure in which its ability to activate CD4⁺ T cells from test and control subjects is assayed. Those peptides that activate CD4⁺ T cells from test subjects but not those from control subjects are identified as peptides that can initiate, promote, or exacerbate the relevant disease or mediate protection from disease or its pathogenic symptoms.

Once such a peptide is identified, it can be synthesized in large amounts, by chemical or recombinant techniques, and used in diagnostic assays similar to the EV procedures listed below. Relevant peptides could be used singly or in combination. Alternatively, expression vectors encoding such a peptide or a combination of such peptides can be used to transfect or transduce appropriate APC (see below), and these can be used in similar diagnostic assays.

Furthermore, multimers (e.g., dimers, trimers, tetramers, pentamers, or hexamers) of a class II MHC molecule complexed with a peptide defined by the method of the invention and conjugated to a detectable label (e.g., a fluorescent moiety, a radionuclide, or an enzyme that catalyzes a reaction resulting in a product that absorbs or emits light of a defined wavelength) can be used to quantify T cells from a subject (e.g., a human patient) bearing cell surface receptors that are specific for such complexes. Relatively high numbers of such T cells are likely to be diagnostic of a relevant disease or an indication that the T cells are involved in immunity to the disease. In addition, continuous monitoring of a patient's relative numbers of multimer-binding T cells can be useful in tracking the course of a disease or the efficacy of therapy. Such assays have been developed using tetramers of class I MHC molecules complexed with an HIV-1-derived or an influenza virus-derived peptide [Altman et al. (1996), Science 274:94-96; Ogg et al. (1998), Science 279:2103-2106], and corresponding class II MHC multimers would be expected to be similarly useful. Such complexes could be produced by chemical cross-linking of purified class II MHC molecules assembled in the presence of a peptide of interest or by modification of already established recombinant techniques for the production of class II MHC molecules containing a single defined peptide [Kazono et al. (1994), Nature 369:151-154; Gauthier et al. (1998), Proc. Natl. Acad. Sci. U.S.A. 95:11828-11833]. The class II MHC molecule monomers of such multimers can be native molecules composed of full-length α and β chains. Alternatively, they can be molecules containing only the extracellular domains of the α and β chains or the α and β chain domains that form the “walls” and “floor” of the peptide-binding cleft.

1.1 IMF

In IMF, the APC is transfected (transiently or stably) with the cDNA encoding the PPI (e.g., CEA) using standard methods known in the art, and high expressing clonal populations are isolated for preparative growth. As used herein, the term “transfected” includes infected. Thus, the cDNA encoding the PPI can be in an expression vector such as a plasmid or an infectious agent such as a virus. Useful viruses include vaccinia virus, measles virus, alpha viruses, and herpes viruses. Additional suitable viruses are listed below in the section describing APL. Infection of the APC can be performed in vitro or in vivo in a mammalian subject prior to harvesting of the cells from the subject. After producing a sufficient number of cells (−1×10⁸ to 1×10¹⁰ depending on the expression of the target tumor antigen), the class II MHC molecules of interest are isolated from the APC by any one of various methods known in the art, e.g., immunoprecipitation. They may be isolated by affinity chromatography by the method of Gorga et al. (1987), J. Biol. Chem. 262:16087-16094.

Peptides bound non-covalently to the isolated class II MHC molecules are then eluted from the latter. A variety of methods known in the art can be used, for example, the method of Chicz et al. (1992), Nature 358:764-768; Chicz and Urban, Immunology Today 15:155-160; Urban et al, Critical Reviews in Immunology 17:387-397 or the novel solid phase extraction protocol as in Example 1.

The eluted peptides are separated by one of a variety of possible chromatographic methods, e.g., reverse phase chromatography. All the resulting fractions that contain peptides are then individually analyzed by MALDI-TOF mass spectrometry, using settings that do not fragment the peptides. The peptides corresponding to all the “peaks” obtained on the MALDI-TOF spectrum can then be subjected to individual amino acid sequence analysis. Alternatively, only novel responses in the “test” cell-line can be subjected to amino acid sequence analysis. Novel responses are peaks that are not observed in a control spectrum generated using a sample of peptides obtained by an identical procedure but omitting the transfection of cells with a plasmid that contains a DNA sequence that encodes the PPI. The sequences of the individual peptides can be obtained by means known to those in the art. They can, for example, be obtained by MALDI-TOF, using instrument settings resulting in the fragmentation of the peptides into small fragments that are analyzed by the mass spectrometer. The amino acid sequences of the peptides are then compared to that of the PPI. Those with a sequence identical to a region of the PPI are candidates for EV.

Alternatively, other approaches for deriving the amino acid sequences of individual peptides can be utilized. These methods invoke a second dimension of peptide separation prior to mass spectrometric analysis. This is often achieved by coupling a separation technique such as reversed phase HPLC to a mass analyzer (such as but not limited to a quadrupole ion trap, triple quadrupole instrument, magnetic sector, Fourier transformation cyclotron resonance, quadropole time-of-flight, or a hybrid of these analyzers) though an electrospray interface. First, novel peptide responses that were detected in the “test” transfected cell-line but not the control can be specifically isolated by the mass spectrometer and fragmented to yield amino acid sequences. Second, fractions can be analyzed in a data dependent mode of operation. In this mode, mass peaks are dynamically and automatically selected for isolation and fragmentation to yield amino acid sequences. No prior knowledge of novel signals is required for this mode of peptide sequencing.

1.2 EV

The EV procedure involves testing of peptides identified by IMF for their ability to (a) bind the class II MHC from which they were eluted and (b) activate various CD4⁺ T cell populations. Peptides with amino acid sequences either identical to those identified by IMF or corresponding to a core sequence derived from a nested group-of peptides identified by the IMF are synthesized. The synthetic peptides are then tested for their ability to bind the class II MHC from which they were eluted and activate CD4⁺ T cells from (a) test subjects expressing the class II MHC molecule of interest and having at least one symptom of the disease; and (b) control subjects expressing the class II MHC molecule of interest and having no symptoms of the disease. Additional control subjects can be those with symptoms of the disease and not expressing the class II MHC molecule of interest. In some diseases (e.g., those with an autoimmune component), responsiveness in the CD4⁺ T cells of test subjects but not in CD4⁺ T cells of the control subjects described in (b) provides confirmatory evidence that the relevant peptide is an epitope that activates CD4⁺ T cells that can initiate, promote, or exacerbate the relevant disease. In other diseases (e.g., cancer or infectious diseases without an autoimmune component), a similar pattern of responsiveness and non-respohsiveness to that described in the previous sentence would indicate that the relevant peptide is an epitope that activates CD4⁺ T cells that can mediate immunity to the disease or, at least, a decrease in the symptoms of the disease.

Absence of a response in subjects with symptoms of the disease but not expressing the class II MHC molecule provides further evidence for the stated activities of the peptide. On the other hand, a response in such control CD4⁺ T cells would not necessarily exclude such a role for the peptide but would suggest that the relevant peptide is capable of (i) binding to some MHC class II molecule expressed by the relevant subject; and (ii) being recognized by CD4⁺ T cells in association with that class II MHC molecule. If these characteristics are confirmed for multiple MHC class II alleles, then the peptide is likely to have widespread utility in patients expressing different class II alleles.

CD4⁺ T cell responses can be measured by a variety of in vitro methods known in the art. For example, whole peripheral blood mononuclear cells (PBMC) can be cultured with and without a candidate synthetic peptide and their proliferative responses measured by, e.g., incorporation of [³H]-thymidine into their DNA. That the proliferating T cells are CD4⁺ T cells can be tested by either eliminating CD4⁺ T cells from the PBMC prior to assay or by adding inhibitory antibodies that bind to the CD4⁺ molecule on the T cells, thereby inhibiting proliferation of the latter. In both cases, the proliferative response will be inhibited only if CD4⁺ T cells are the proliferating cells.

Alternatively, CD4⁺ T cells can be purified from PBMC and tested for proliferative responses to the peptides in the presence of APC expressing the appropriate class II MHC molecule. Such APC can be B-lymphocytes, monocytes, macrophages, or dendritic cells, or whole PBMC. APC can also be immortalized cell lines derived from B-lymphocytes, monocytes, macrophages, or dendritic cells. The APC can endogenously express the class II MHC molecule of interest or they can express transfected polynucleotides encoding such molecules. Where the subjects are humans, the APC can also be T cells since human T cells are capable of expressing class II MHC molecules. In all cases the APC can, prior to the assay, be rendered non-proliferative by treatment with, e.g., ionizing radiation or mitomycin-C.

As an alternative to measuring cell proliferation, cytokine production by the CD4⁺ T cells can be measured by procedures known to those in art. Cytokines include, without limitation, interleukin-2 (IL-2), IFN-γ, IL-4, IL-5, TNF-α, interleukin-3 (IL-3), interleukin-6 (IL-6), interleukin-10 (IL-10), interleukin-12 (IL-12), GM-CSF, RANTES, MIP-1α, MIP-1β and transforming growth factor β (TGFβ). Assays to measure them include, without limitation, ELISA, ELISPOT and bio-assays in which cells responsive to the relevant cytokine are tested for responsiveness (e.g., proliferation) in the presence of a test sample. Alternatively, cytokine production by CD4⁺ lymphocytes can be directly visualized by intracellular immunofluorescence staining and flow cytometry.

Once peptide epitopes associated with a particular disease have been identified, the EV described above can be used as a diagnostic test for the disease. Thus, lymphocytes from a subject suspected of having or being susceptible to the disease can be tested by any of the described methods for a CD4⁺ T lymphocyte response to one or more (e.g., 2, 3, 4, 5, 6, 10, 15, or 20) appropriate peptides. If a significant CD4⁺ T lymphocyte is detected, it is likely that the subject has or will develop the disease. The disease can be, for example, cancer and the peptides can be derived from, for example, CEA. Appropriate peptides can be, for example, any of those listed below (e.g., those with SEQ ID NOS:1-7 and 13-15).

As an alternative to the above-described EV, peptides identified by the IMF can be tested for their ability to bind to an appropriate class II MHC molecule by methods known in the art using, for example, isolated class II MHC molecules or cells transfected with nucleic acid molecules encoding class II MHC molecules. One such method is described in Example 2. These binding assays can also be used to test the ability of the peptides to bind to alternative class II MHC molecules, i.e., class II MHC molecules other than those from which they were eluted using the IMF method of the invention. Once such alternative class II MHC molecules are shown to bind the peptide(s), the diagnostic methods of the invention (using such peptides) and therapeutic methods of the invention (using either the peptides or APL derived from them) can be applied to subjects expressing such alternative class II MHC molecules.

1.3 Diseases and Their Associations with Class II MHC Genes

The methods of the invention can be applied to the analysis of peptides involved in diseases associated with expression of defined class II MHC molecules and in which pathology or protection is due to the action of activated CD4⁺ T cells. Such diseases include, without limitation, certain infectious diseases, cancer, and autoimmune diseases. Three are discussed below.

An example of an infectious disease that fulfills the above criteria is human leprosy, which is caused by Mycobacterium leprae. The bacteria infect and thrive in peripheral Schwann cells and macrophages. The disease is characterized by depressed cellular immunity but normal antibody responses. Leprosy has been associated with the expression of DRB1 class II MHC molecules in which codon 13 encodes Arg or codons 70 and 71 encode Arg [Zerva et al. (1996), J. Exp. Med. 183: 829-836].

The ability to spontaneously clear hepatitis C virus is associated with expression of DQB1*0301 molecules. Since DQB1*0302 is under-represented in hepatitis virus C infected subjects, DQB1*0302 expressing individuals may be protected from infection with the virus [Cramp et al. (1998), J. Hepatol. 29:207-213].

Melanoma cell-specific CD4⁺ T cells, which may be-involved in protective immune responses to malignant melanoma, recognize tyrosinase epitopes presented by HLA-DRB1*0401 class II molecules [Topalian et al. (1996), supra]. With respect to cancers, the reticular cell sarcomas of SJL mice are dependent for growth on cytokines produced by activated CD4⁺ T cells and require the expression of certain class II MHC molecules.

Examples of autoimmune diseases to which the methods of the invention can be applied include, without limitation, IDDM, rheumatoid arthritis (RA), multiple sclerosis (MS), systemic lupus erythmatosus (SLE), and myasthenia gravis (MG). RA is associated with expression of DRB1 alleles (DRB1*0101, 0401, 0403, and 0405). MS is associated with expression of DRB1*1501, DQA1*0102 and DQB1*0602 alleles. SLE is associated with the expression of DRB1*03, DRB1*1501, DQA1*0501 and DQB1*0201 alleles. MG is associated with the expression of DR3 and DQ2 (DQA1*0501-DQB1*0201 and DQA1*0201-DQB1*0201) alleles. Autoimmune ovarian failure is associated with DQB1 genes encoding Asp at position 57. Graves' thyroiditis, Hashimoto's thyroiditis, and primary hypothyroidism all show weak association with the expression of the DR5 and DR3 alleles. Coeliac disease is associated with the expression of HLA-DQA1*0501 and DQB1*0201 alleles. Primary biliary cirrhosis is associated with the expression of DRB1*0801-DQA1*0401/0601-DQB1*04 alleles. Autoimmune hepatitis is associated with the expression of DRB3*0101 and DRB1*0401 alleles. Addison's disease is associated with the expression of DRB1*03, DQA1*0501 and DQB1*0201 alleles. Vitiligo is associated with the expression of DRB1*0701 and DQ2 alleles. Anti-glomerular basement membrane disease (Goodpasture's syndrome) is associated with the expression of DR15 and DR4 alleles.

Pathology in RA, MS, and IDDM is considered to be due predominantly to CD4⁺ T cell-dependent, cell-mediated autoimmune responses, while that of SLE and MG is due predominantly to CD4⁺ T cell-dependent, antibody-mediated autoimmune responses. In RA the inflammatory response induced by the activated CD4⁺ T cells is focused on joint synovia, in MS on neural myelin sheaths, and in IDDM on pancreatic β cells located in the islets of Langerhans. SLE is a systemic autoimmune disease involving multiple organs. The muscle fatigue observed in MG is due to the development in the patient of antibodies that bind to the acetylcholine receptor in neuromuscular junctions.

Other MHC class-II associated diseases are listed above.

1.4 Species

The methods of the invention can be applied to diseases with the described characteristics in a wide range of mammalian species, e.g., humans, non-human primates, horses, cattle, pigs, sheep, goats, dogs, cats, rabbits, guinea pigs, hamsters, rats, and mice. They will preferably be applied to diseases of humans.

1.5 Class II MHC Molecules

Class II MHC molecules have been identified in multiple mammalian species. In some of these species, expression of a particular class II MHC molecule has been associated with a particular CD4⁺ T cell-mediated diseases (see above). In humans, for example, the class II MHC molecules are designated HLA-DR, HLA-DQ, and HLA-DP and in mice, H-2A and H-2E. In all species, there are multiple alleles of each gene.

1.6 Antigen Presenting Cells (APC)

APC that can be used for the IMF methods of the invention can be any mammalian cell that expresses class II MHC molecules on its surface, e.g., those listed above for use in EV (B lymphocytes, macrophages, monocytes, dendritic cells, and, in humans, T cells). APC can also be tumor cells, e.g., B cell lymphoma cells or melanoma cells. It is also not required that the cells constitutively express class II MHC molecules. Class II MHC can be induced (in vitro or in vivo) (e.g., by IFN-γ) in such cells. Alternatively, immortalized lines of such cells can be used.

1.7 Polypeptide Antigens

Polypeptide antigens that can be used with the IMF methods can be those with a known amino acid sequence or those in which at least part of the amino acid sequence is known. They can be polypeptides that themselves are known or suspected to be involved in the disease process or immunity to the disease (e.g., CEA in cancer) or they can be derived from microbial organisms known or suspected to be involved in the disease process (e.g., M. leprae in leprosy). Examples of other polypeptide antigens include the core and viral coat proteins of viruses such as hepatitis C virus, the heat shock proteins of mycobacteria, and tyrosinase in melanoma. Furthermore, the polypeptide antigen can be the full-length protein or it can be a fragment of the protein known or suspected to be involved in the disease process.

2. Peptides

Peptides of the invention include, but are not limited to, peptides that bind to class II MHC molecules and activate CD4⁺ T cells involved in a disease process or protection from a disease. The class II MHC molecule can be a class II MHC molecule that is associated with susceptibility or resistance to a disease. Diseases can be any of the diseases cited herein and the species from which the class II MHC molecules and/or peptides are obtained can be any of those cited herein. The class II MHC molecules are preferably human class II HLA molecules, i.e., DR, DP or DQ molecules. The peptides can be, for example, peptides that bind to DR4 or DR1 molecules. The polypeptides from which the peptides of the invention are derived can be any of those cited herein. The peptides generally are 9 to 30 (e.g., 13 to 25) amino acids in length.

The peptides can be derived, for example, from CEA and can bind to HLA-DR1 or HLA-DR4 molecules. The peptides can be, for example, any one of the following peptides:

KEVLLLVHNLPQH (SEQ ID NO:1); KEVLLLVHNLPQHL (SEQ ID NO:2;) KEVLLLVHNLPQHLF (SEQ ID NO:3); KEVLLLVHNLPQHLFG (SEQ ID NO:4); GKEVLLLVHNLPQHL (SEQ ID NO:5); EGKEVLLLVHNLPQHLFG (SEQ ID NO:6); YLWWVNGQSLPVSPR (SEQ ID NO:7); EGKEVLLLVHNLPQHL (SEQ ID NO:13); NPPAQYSWLIDGNIQQH (SEQ ID NO:14); or NPPAQYSWLIDGNIQQHT (SEQ ID NO:15). Also included are peptides containing any of the above sequences, plus 1-15 residues on either end or 1-15 residues on each end.

In addition, the peptides can be isolated fragments of CEA which are shorter than full-length CEA. In such peptides, the fragments can contain one or more (e.g., one two, three, four, five, six, seven, eight, nine, or ten) amino acid sequences which can be, for example, those amino acid sequences with SEQ ID NOS:1-7 and 13-15.

The peptides can also be peptides that include one or more CEA segments (e.g., SEQ ID NOS:1-7 and 13-15) and one or more (e.g., one, two, three, four, five, six, seven, eight, nine, ten, 11, 12, 13, 14, 15, 16, 17, 18, 19, 20, 21, 22, 23, 24, 25, 30, 35, 40, 50, 55, 60, 65, 70, 75, 80, 90, 100, 150, 200, 250, 300, 400, 500, or more) amino acids at the C-terminus, the N-terminus, or both the C-terminus and the N-terminus of the CEA segment. The additional amino acid residues can be CEA or non-CEA amino acids. The peptide will, however, not be identical to full-length CEA.

In addition, peptides identified as being associated with any of the diseases listed herein (e.g., cancer such as colon cancer or any of the infectious diseases recited herein) can be used to activate lymphocytes (e.g., CD4⁺ lymphocytes) that cause, directly or indirectly, the death of pathogenic target cells such as cancer cells or pathogen-infected cells. Alternatively, in other diseases such as the autoimmune diseases recited herein, the peptides can be used to induce immunological tolerance in lymphocytes (e.g., CD4⁺ T lymphocytes) associated with the initiation, progress, or pathological symptoms of the disease. Tolerization of these lymphocytes can be useful for prophylaxis against and/or therapy of the relevant disease. Lymphocyte activation or tolerization can be achieved by administering an appropriate peptide to a subject. Methods of testing for efficacy of a peptide in activating or in inducing tolerance in lymphocytes (e.g., CD4+ lymphocytes), methods and routes of administration, and doses to be administered are essentially the same as those described below for APL. Furthermore, the peptides can be modified in any of the ways described below for APL. In addition, peptidomimetic forms of the peptides can be produced by methods known in the art (see below in section on APL). The peptides can be fragments of any the polypeptides disclosed herein, e.g., CEA, PSA, insulin, proinsulin, preproinsulin, GAD65, IA-2, or phogrin. They can be, for example, those with SEQ ID NOS:1-7 and 13-15 or any of those described above.

The invention also includes a method of activating or tolerizing CD4⁺ T cells in which full-length CEA or DNA encoding full-length CEA is administered to a mammal. Following this administration, CD4⁺ T cells of the mammal are tested for responsiveness to a naturally processed fragment of CEA. Such a fragment can be, but is not limited to, a fragment with any of SEQ ID NOS:1-7 and 13-15.

The CEA peptides of the invention (e.g., those with SEQ ID NOS:1-7 and 13-15) can be used for purposes other than therapy. They can be used, for example, in diagnostic assays and for methods of screening for reagents that bind to complexes of class II MHC molecules and CEA peptides (see below).

The peptides can be prepared using the described IMF methodologies. Smaller peptides (fewer than about 50 amino acids long) can also be conveniently synthesized by standard chemical means. In addition, both polypeptides and peptides can be produced by standard in vitro recombinant DNA techniques, and in vivo transgenesis using the nucleotide sequences encoding the appropriate polypeptides, peptides or APL. Methods well known to those skilled in the art can be used to construct expression vectors containing relevant coding sequences and appropriate transcriptional/translational control signals. See, for example, the techniques described in Sambrook et al., Molecular Cloning: A Laboratory Manual, Cold Spring Harbor Laboratory, N.Y., 1989, and Ausubel et al., Current Protocols in Molecular Biology, Green Publishing Associates and Wiley Interscience, N.Y., 1989.

The invention also features isolated nucleic acid molecules encoding the peptides of the invention. These nucleic acid molecules can be cDNA, genomic DNA, synthetic DNA, or RNA, and can be double-stranded or single-stranded (i.e., either a sense or an antisense strand). Segments of these molecules are also considered within the scope of the invention, and can be produced, for example, by the polymerase chain reaction (PCR) or generated by treatment with one or more restriction endonucleases. A ribonucleic acid (RNA) molecule can be produced by in vitro transcription. Preferably, the nucleic acid molecules encode peptides that, regardless of length, are soluble under normal physiological conditions.

The nucleic acid molecules of the invention can contain naturally occurring sequences, or sequences that differ from those that occur naturally, but, due to the degeneracy of the genetic code, encode the same polypeptide (for example, the peptides with SEQ ID NOS:1-7 and 13-15). In addition, these nucleic acid molecules are not limited to coding sequences, e.g., they can include some or all of the non-coding sequences that lie upstream or downstream from a coding sequence.

The nucleic acid molecules of the invention can be synthesized (for example, by phosphoramidite-based synthesis) or obtained from a biological cell, such as the cell of a mammal. Thus, the nucleic acids can be those of a human, non-human primate (e.g., monkey), mouse, rat, guinea pig, cow, sheep, horse, pig, rabbit, dog, or cat.

In addition, the isolated nucleic acid molecules of the invention encompass segments that are not found as such in the natural state. Thus, the invention encompasses recombinant nucleic acid molecules (for example, isolated nucleic acid molecules encoding any of the peptides described herein) incorporated into a vector (for example, a plasmid or viral vector) or into the genome of a heterologous cell (or the genome of a homologous cell, at a position other than the natural chromosomal location).

Techniques associated with detection or regulation of genes are well known to skilled artisans and such techniques can be used to diagnose and/or treat disorders associated with aberrant CEA expression, e.g., colon cancer. Hybridization can be used as a measure of homology between two nucleic acid sequences. Thus a nucleic acid encoding a peptide of the invention (e.g., a CEA peptide such as a peptide with SEQ ID NOs: 1-7 or 13-15), or a portion of such a nucleic acid, can be used as hybridization probe according to standard hybridization techniques. The hybridization of a CEA peptide probe to DNA or RNA from a test source (e.g., a mammalian cell) is an indication of the presence of CEA DNA or RNA, respectively, in the test source. Hybridization conditions are known to those skilled in the art and can be found in Current Protocols in Molecular Biology, John Wiley & Sons, N.Y., 6.3.1-6.3.6, 1991. Moderate hybridization conditions are defined as equivalent to hybridization in 2× sodium chloride/sodium citrate (SSC) at 30° C., followed by one or more washes in 1×SSC, 0.1% SDS at 50-60° C. Highly stringent conditions are defined as equivalent to hybridization in 6× sodium chloride/sodium citrate (SSC) at 45° C., followed by one or more washes in 0.2×SSC, 0.1% SDS at 50-65° C.

The invention also encompasses: (a) vectors that contain any of the foregoing peptide coding sequences and/or their complements (that is, “antisense” sequence); (b) expression vectors that contain any of the foregoing peptide coding sequences operatively associated with any transcriptional/translatiorial regulatory element (examples of which are given below in the section on APL) necessary to direct expression of the coding sequences; (c) expression vectors containing, in addition to sequences encoding a peptide of the invention, nucleic acid sequences that are unrelated to nucleic acid sequences encoding the peptide of the invention, such as nucleic acid sequences encoding a reporter, marker, or a signal peptide (e.g., a heterologous signal peptide); and (d) genetically engineered host cells that contain any of the foregoing expression vectors and thereby express the nucleic acid molecules of the invention.

Where the nucleic acids form part of a hybrid gene encoding additional polypeptide sequences, for example, sequences that function as a marker or reporter, markers or reporter genes include β-lactamase, chloramphenicol acetyltransferase (CAT), adenosine deaminase (ADA), aminoglycoside phosphotransferase (neo^{r}, G418r), dihydrofolate reductase (DHFR), hygromycin-B-phosphotransferase (HPH), thymidine kinase (TK), lacZ (encoding β-galactosidase), and xanthine guanine phosphoribosyltransferase (XGPRT). As with many of the standard procedures associated with the practice of the invention, skilled artisans will be aware of additional useful reagents, for example, additional sequences that can serve the function of a marker or reporter. Generally, the hybrid polypeptide will include a first portion and a second portion, the first portion being a peptide of the invention (e.g., a peptide with any of SEQ ID NOS:1-7 or 13-15) and the second portion being, for example, an immunoglobulin constant region.

A variety of host-expression vector systems can be used to express the peptides and polypeptides. Such host-expression systems represent vehicles by which the polypeptides of interest can be produced and subsequently purified, but also represent cells that can, when transformed or transfected with the appropriate nucleotide coding sequences, produce the relevant peptide or polypeptide in situ. These include, but are not limited to, microorganisms such as bacteria, e.g., E. coli or B. subtilis, transformed with recombinant bacteriophage DNA, plasmid or cosmid DNA expression vectors containing polypeptide coding sequences; yeast, e.g., Saccharomyces or Pichia, transformed with recombinant yeast expression vectors containing the appropriate coding sequences; insect cell systems infected with recombinant virus expression vectors, e.g., baculovirus; plant cell systems infected with recombinant virus expression vectors, e.g., cauliflower mosaic virus (CaMV) or tobacco mosaic virus (TMV), or transformed with recombinant plasmid expression vectors, e.g., Ti plasmids, containing the appropriate coding sequences; or mammalian cell systems, e.g., COS, CHO, BHK, 293 or 3T3, harboring recombinant expression constructs containing promoters derived from the genome of mammalian cells, e.g., metallothionein promoter, or from mammalian viruses, e.g., the adenovirus late promoter or the vaccinia virus 7.5K promoter.

3. APL

An altered peptide ligand (APL) is a variant peptide in which 1-6 (i.e., 1, 2, 3, 4, 5, or 6) amino acid residues of a parent wild-type peptide that activates a response in CD4⁺ T cells have been changed. In an APL of the invention, fewer than half of the residues of the wild-type peptide are changed, e.g., fewer than 50%, fewer than 40%, fewer than 30%, fewer than 25%, fewer than 20%, fewer than 15%, fewer than 10%, or fewer than 5%. Thus, for example, in an APL derived from a wild-type peptide 20 amino acids long and differing from the wild-type peptide at 6 positions, 30% of the amino acids of the wild-type peptide are changed. Alternatively, in an APL derived from a wild-type peptide 15 amino acids long and differing from the wild-type peptide at 3 positions, 20% of the amino acids of the wild-type peptide are changed.

An APL retains at least some ability to bind to the class II MHC molecule to which the parent peptide binds and at least some ability to be recognized by the antigen-specific T lymphocyte receptor(s) of the CD4⁺ T cell(s) that recognize the parent peptide bound to the same type of class II MHC molecule. However, by definition, the APL activates a response in the CD4⁺ T cells that is qualitatively different from that activated by the parent peptide. For example, while the parent peptide can activate a helper T cell 1-(Th1-)type response in which the cytokines interleukin-2 (IL-2), interferon-γ (IFN-γ), and tumor necrosis factor-α (TNF-α) are produced by the activated CD4⁺ T cells, an APL derived from this parent peptide might instead activate a helper T cell 2- (Th2-) type response in the CD4⁺ T cells. In a Th2 response, the cytokines interleukin-4 (IL-4), interleukin-5 (IL-5), and interleukin-10 (IL-10) are produced by the activated CD4⁺ T cells. Alternatively, if a particular parent peptide elicits a Th2 response in a given CD4⁺ T cell, an APL derived from the parent peptide could activate a Thi response in the T cell. Some APL have been shown to switch a Th1 response to a Th0 response in which both Th1- and Th2-type cytokines are produced. Furthermore, an APL could redirect a CD4⁺ T cell response towards a Th3-type response in which the predominant cytokine produced is transforming growth factor-β (TGF-β). TGF-β has been shown to be suppressive of a wide range of immune responses.

In general, Th1 responses are associated with cell-mediated immune responses and Th2 responses are associated with antibody—(i.e., B cell-)mediated immune responses. Thus, the relative number of CD4⁺ T cells responding in a Th1—versus a Th2-type fashion will determine the nature (cell-mediated versus antibody-mediated) of the immune response generated by an antigen in a particular individual.

Some conditions, and in particular autoimmune diseases (e.g., RA, IDDM, and MS), have been shown to be due to cellular immune responses and thus to be dependent on Th1 CD4⁺ T cell responses. Other diseases (e.g., MG and SLE) have been shown to be mediated by antibody (i.e., B-cell) responses, and thus to be dependent on Th2 CD4⁺ T cell responses. Thus, an APL that serves to direct a CD4⁺ T cell response from a Th1 to a Th2 response can be useful in treatment or prevention of the first category of diseases and an APL that serves to alter a CD4⁺ T cell response in a Th2 to Th1 direction can be useful in the treatment or prevention of the second category of diseases.

The amino acid substitutions in APL can be radical. For example, an amino acid with a positively charged side chain (e.g., lysine) can be replaced by an amino acid with a negatively charged side chain (e.g., aspartic acid) or a hydrophobic side chain (e.g., isoleucine) and vice versa. In addition, an amino acid with a bulky side chain (e.g., tryptophan) can be replaced with an amino acid with small side chain (e.g., glycine or alanine) and vice versa. Alternatively, the substitutions can be conservative. For example, a negatively charged amino acid can be replaced with another negatively charged amino acid (e.g., aspartic acid with glutamic acid) or one hydrophobic amino acid with another hydrophobic amino acid (e.g., leucine with valine or isoleucine).

Methods to test whether a given APL elicits a predominantly Th1, Th2, Th3, Th0, or other CD4⁺ T cell response are known in the art. In brief, an APL of interest can be administered to a test subject (e.g., a mouse) expressing a class II MHC molecule of interest (e.g., a human class II MHC molecule) by any one of a variety of routes, e.g., intramuscular, intravenous, subcutaneous, intradermal, intraperitoneal, intrarectal, intravaginal, intranasal, intragastric, intratracheal, or intrapulmonary. In addition, administration can be oral or transdermal, employing a penetrant such as a bile salt, a fusidic acid or another detergent. The injections can be single or multiple (e.g., 2-, 3-, 4-, 6-, 8-, or 10-fold). The peptide can be administered in a physiologically acceptable solution (e.g., a saline solution) and can be administered with or without an adjuvant (e.g., Freund's complete or incomplete adjuvant or cholera toxin). After immunization, the animal can be challenged with the APL, either in vivo or in vitro, by methods known in the art, and the levels of individual cytokines produced measured. In the case of an in vivo challenge, the cytokine secreted into the blood or some other bodily fluid (e.g., urine, saliva, or semen) or lavage (e.g., nasal, pulmonary, rectal, gastric, or vaginal lavages) can be measured. Alternatively, lymphoid cells can be isolated from the animal after challenge and the level of cytokines produced by the cells can be tested, e.g., by culturing and measurement of cytokine. levels in culture supernatant by, e.g., ELISA. Isolated lymphoid cells can also be tested for relative numbers of cells producing the cytokines by assays such as the ELISPOT assay or fluorescence analysis following intracellular staining with one or more cytokine binding antibodies, each conjugated with a different fluorophore which emits light of a distinct wavelength. Fluorophores fluorescing at different wavelengths (i.e., colors) are known in the art. Using such fluorescence assays, it is possible to ascertain the range of cytokines being produced by a single cell. If the lymphoid cells are challenged in vitro, assays such as the ELISPOT assay or ELISA can be used. Should immunization and challenge with an APL result in relatively low levels of IL-2, IFN-γ, and TNF-α and relatively high levels of IL-4, IL-5, and IL-10, while immunization and challenge with the parental peptide results in the inverse pattern, the conclusion would be that the APL is useful for switching a response from a Th1 to a Th2 pattern of cytokine production. Where the Th1 response is pathogenic, treatment with the APL can be therapeutic or prophylactic. Similarly, APL and their parent peptides can be tested for their relative abilities to shift a response from a Th2 type response to a Th1 type response, or from a Th1-type response to a Th0- or a Th3-type response. The ability of an APL to activate responses such as CD4⁺ CTL responses, anti-angiogenic responses, or macrophage/eosinophil activating responses can be determined by methods familiar to those in the art.

APL can also be tested by any of the protocols in human volunteers. Alternatively, lymphoid cells could be isolated from a subject (e.g., a human subject) and both immunized and challenged in vitro. In addition, APL can be administered to “SCID-Hu” mice, which are mice genetically deficient in murine T and B lymphocytes and reconstituted with human lymphoid cells. Due to the inherent immunological deficiency in these animals, the human lymphoid cells are not rejected and will engraft. After immunizing and challenging these mice with an APL (using any of methodologies described above), their cytokine responses can be measured by any of the methods described above. To ensure that the cytokines detected in the assays are of human origin, human species-specific reagents (e.g., antibodies) can be used for the assays (e.g., ELISA or ELISPOT). Furthermore, in order to exclude presentation of the APL to human CD4⁺ T cells by murine class II MHC molecules, SCID mice could be bred with class II MHC “knockout” mice in order to generate mice deficient in both lymphocytes and murine class II MHC molecules. By reconstituting the resulting mice with human lymphoid cells, a SCID-Hu mouse is provided in which essentially the only CD4⁺ T cells capable of responding are human CD4⁺ T cells and the only class II MHC molecules capable of presenting the APL are human class II MHC molecules on the surface of human lymphoid cells. Alternatively, the recipient of human lymphoid cells could be a hybrid mouse derived by breeding SCID mice with DR (e.g., DR4) or DQ (e.g., DQ8) transgenic mice made on a class II MHC knockout background. Again the only class II MHC molecules present would be the human DR or DQ molecules contributed by the transgenic parental mice.

RAG-1 deficient mice can be used instead of the SCID mice for generation of the described human/mouse chimeric animals. RAG-1 deficient mice, like SCID mice, lack T and B lymphocytes but have the advantage that the relevant mutation is not “leaky.” Thus, while late in life SCID mice can develop a low number of lymphocytes, this does not occur in RAG-1 deficient mice.

The APL of the invention can be obtained by any of the methods described above for peptides and polypeptides. APL of the invention also include those described above, but modified for in vivo use by the addition, at either or both the amino- and carboxyl-terminal ends, of a blocking agent to facilitate survival of the relevant peptide in vivo. This can be useful in those situations in which the peptide termini tend to be degraded by proteases in vivo.

Alternatively, blocking agents such as pyroglutamic acid or other molecules known in the art can be attached to the amino and/or carboxyl terminal residues, or the amino group at the amino terminus or carboxyl group at the carboxyl terminus can be replaced with a different moiety. Likewise, the peptides can be covalently or noncovalently coupled to pharmaceutically acceptable “carrier” proteins prior to administration.

Also of interest are peptidomimetic compounds that are designed based upon the amino acid sequences of the APL. Peptidomimetic compounds are synthetic, non-peptide compounds having a three-dimensional conformation (i.e., a “peptide motif”) that is substantially the same as the three-dimensional conformation of a selected peptide. The peptide motif provides the peptidomimetic compound with the ability to activate CD4⁺ T cells in a manner qualitatively identical to that of the APL from which the peptidomimetic was derived. Peptidomimetic compounds can have additional characteristics that enhance their therapeutic utility, such as increased cell permeability and prolonged biological half-life.

The peptidomimetics typically have a backbone that is partially or completely non-peptide, but with side groups that are identical to the side groups of the amino acid residues that occur in the peptide on which the peptidomimetic is based. Several types of chemical bonds, e.g., ester, thioester, thioamide, retroamide, reduced carbonyl, dimethylene and ketomethylene bonds, are known in the art to be generally useful substitutes for peptide bonds in the construction of protease-resistant peptidomimetics.

4. Methods of Therapy Using APL

An APL that has the ability to elicit a cytokine response in CD4⁺ T cells that is non-pathogenic and/or is suppressive of a pathogenic CD4⁺ T cell cytokine response elicited by the APL's parental peptide, could be useful in therapy, palliation, or prophylaxis of a disease caused by the pathogenic CD4⁺ T lymphocyte response to the parental peptide.

For example, if “peptide x” elicits a potent Th1-type response in CD4⁺ T cells in a patient with a HLA-DR4, treatment of the patient with a peptide x-derived APL that elicits a Th2 CD4⁺ T cell response can be therapeutic or palliative in that patient. Alternately, if peptide x elicits a Th1 response, an APL derived from peptide x could elicit a CD4⁺ T-cell response that enhances (a) migration of CD8+tumoricidal CTL to the vicinity of the tumor cells for identification and specific killing of the tumor cells; or (b) migration of tumoricidal macrophages and eosinophils to the vicinity of the tumor cells and thereby enhance killing of the tumor cells. Alternatively, an APL could elicit an anti-angiogeneic response in the microenvironment of the tumor that cuts off the nutrient supply needed for continued tumor cell division and consequent tumor growth. Furthermore, an APL derived from a tumor antigen peptide could activate a CD4⁺ CTL response more effectively than the parent peptide from which the APL was derived.

These methods of the invention fall into 2 basic classes, i.e., those using in vivo approaches and those using ex vivo approaches.

4.1 In Vivo Approaches

In one in vivo approach, the APL (peptide or peptidomimetic) itself is administered to the subject by any of the routes listed above. It is preferably delivered directly to an appropriate lymphoid tissue (e.g. spleen, lymph node, or mucosal-associated lymphoid tissue (MALT)).

The dosage required depends on the choice of APL, the route of administration, the nature of the formulation, the nature of the patient's illness, and the judgment of the attending physician. Suitable dosages are in the range of 0.1-100.0 μg/kg. Wide variations in the needed dosage are to be expected in view of the variety of APL available and the differing efficiencies of various routes of administration. For example, oral administration would be expected to require higher dosages than administration by i.v. injection. Variations in these dosage levels can be adjusted using standard empirical routines for optimization, as is well understood in the art.

Alternatively, a polynucleotide containing a “minigene” encoding the APL can be delivered to an appropriate cell of the animal. Expression of the minigene will preferably be directed to lymphoid tissue of the subject by, for example, delivery of the polynucleotide to the lymphoid tissue. This can be achieved by, for example, the use of a polymeric, biodegradable microsphere, microparticle or microcapsule delivery vehicle, sized to optimize phagocytosis by phagocytic cells such as macrophages. For example, PLGA (poly-lacto-co-glycolide) microparticles approximately 1-10 μm in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm). Microparticles useful for nucleic acid delivery, methods for making them, and methods of use are described in greater detail in U.S. Pat. No. 5,783,567, incorporated herein by reference in its entirety. Microparticles may also be made, for example, by the methods of Mathiowitz et al. (WO 95/2429), incorporated herein by reference in its entirety.

Alternatively, a polynucleotide containing a “minigene” encoding the APL can be delivered in a pharmaceutically acceptable carrier such as saline, lipids, liposomes, particulates, virus-like particles, or nanospheres; as colloidal suspensions; or as powders. The nucleic acid can be naked or associated or complexed with a delivery vehicle. For a description of the use of naked DNA, see, e.g., U.S. Pat. No. 5,693,622. Nucleic acids and polypeptides can be delivered using delivery vehicles known in the art, such as ISCOMS, microspheres, microcapsules, microparticles, gold particles, virus-like particles, nanoparticles, polymers, condensing agents, polysaccharides, polyamino acids, dendrimers, saponins, adsorption enhancing materials, or fatty acids. Viral particles can also be used, e.g., retroviruses, adenovirus, adeno-associated virus, pox viruses, SV40 virus, alpha virus or herpes viruses.

The vectors can be incorporated alone into these delivery vehicles or co-incorporated with tissue-specific antibodies. Alternatively, one can prepare a molecular conjugate composed of a plasmid or other vector attached to poly-L-lysine by electrostatic or covalent forces. Poly-L-lysine binds to a ligand that can bind to a receptor on target cells [Cristiano et al. (1995), J. Mol. Med. 73:479]. Alternatively, lymphoid tissue specific targeting can be achieved by the use of lymphoid tissue-specific transcriptional regulatory elements (TRE) such as a B lymphocyte, T lymphocyte, or, optimally, dendritic cell specific TRE. Lymphoid tissue specific TRE are known [Thompson et al. (1992), Mol. Cell. Biol. 12:1043-1053; Todd et al. (1993), J. Exp. Med. 177:1663-1674; Penix et al. (1993), J. Exp. Med. 178:1483-1496].

The nucleic acid sequence encoding an APL of interest with an initiator methionine and optionally a trafficking sequence is operatively linked to a promoter or enhancer-promoter combination in the expression vector.

Short amino acid sequences can act as signals to target proteins to specific intracellular compartments. For example, hydrophobic signal peptides (e.g., MAISGVPVLGFFIIAVLMSAQESWA, SEQ ID NO:8) are typically found at the amino terminus of proteins destined for the ER. The sequence KFERQ (SEQ ID NO:9)(and other closely related sequences) is known to target intracellular polypeptides to lysosomes, while other sequences (e.g., MDDQRDLISNNEQLP, SEQ ID NO:10) target polypeptides to endosomes. In addition, the peptide sequence KDEL (SEQ ID NO:11) has been shown to act as a retention signal for the ER. Each of these signal peptides, or a combination thereof, can be used to traffic the APL of the invention as desired. For example, a construct encoding a given APL linked to an ER-targeting signal peptide would direct the peptide to the ER, where it would bind to the class II MHC molecule as it is assembled, preventing the binding of intact invariant chain (Ii) which is essential for trafficking. Alternatively, a construct can be made in which an ER retention signal on the APL would help prevent the class II MHC molecule from ever leaving the ER. If instead an APL of the invention is targeted to the endosomic compartment, this would ensure that large quantities of the APL are present at the site of class II MHC to peptide complexing, thereby increasing the likelihood that the peptide incorporated into the class II MHC complex is the APL of the invention rather than a naturally-occurring, irrelevant peptide. The likelihood of APL being available for incorporation into class II MHC can be increased by linking the APL to an intact Ii polypeptide sequence. Since Ii is known to traffic class II MHC molecules to the endosomes, the hybrid Ii would carry one or more copies of the APL along with the class II MHC molecule; once in the endosome, the hybrid Ii would be degraded by normal endosomal processes to yield both multiple copies of the APL (or peptides similar to it), and an open class II MHC peptide binding cleft. DNAs encoding APL linked to targeting signals can be generated by PCR or other standard genetic engineering or synthetic techniques. Trafficking sequences are described in greater detail in U.S. Pat. No. 5,827,516 incorporated herein by reference in its entirety.

A promoter is a TRE composed of a region of a DNA molecule, typically located within 100 nucleotide pairs upstream of the point at which transcription starts. Enhancers provide expression specificity in terms of time, location, and level. Unlike a promoter, an enhancer can function when located at variable distances from the transcription initiation site, provided a promoter is present. An enhancer can also be located downstream of the transcription initiation site. The coding sequence of the expression vector is operatively linked to a transcription terminating region.

Suitable expression vectors include plasmids and viral vectors such as herpes viruses, retroviruses, vaccinia viruses, attenuated vaccinia viruses, canary pox viruses, adenoviruses and adeno-associated viruses, among others.

Polynucleotides can be administered in a pharmaceutically acceptable carrier. Pharmaceutically acceptable carriers are biologically compatible vehicles which are suitable for administration to a human, e.g., physiological saline. A therapeutically effective amount is an amount of the polynucleotide which is capable of producing a medically desirable result in a treated animal. As is well known in the medical arts, the dosage for any one patient depends upon many factors, including the patient's size, body surface area, age, the particular compound to be administered, sex, time and route of administration, general health, and other drugs being administered concurrently. Dosages will vary, but a preferred dosage for administration of polynucleotide is from approximately 10⁶ to 10¹² copies of the polynucleotide molecule. This dose can be repeatedly administered, as needed. Routes of administration can be any of those listed above.

4.2 Ex Vivo Approaches

In one ex vivo approach, lymphoid cells, including CD4⁺ T lymphocytes, are isolated from the subject and exposed to the APL in vitro. The lymphoid cells can be exposed once or multiply (e.g., 2, 3, 4, 6, 8, or 10 times). The pattern of cytokine production by the lymphoid cells can be tested after one or more exposures. Once the desired cytokines are being produced by the lymphoid cells, they are reintroduced into the subject via any of the routes listed herein. The therapeutic or prophylactic efficacy of this ex vivo approach is dependent on the ability of the ex vivo APL-activated lymphocytes to actively suppress a pathogenic CD4⁺ T cell response to the parental wild-type peptide. The potential value of such an approach is indicated by experiments in which CD4⁺ T cells producing Th2- (or Th0- or Th3-)type cytokines actively suppressed ongoing Th1 responses and disease caused by such Th1 responses [Nicholson and Kuchroo (1996), Curr. Opinion in Immunol. 8:837-842].

An alternative ex vivo strategy can involve transfecting or transducing cells obtained from the subject with a polynucleotide containing the APL-encoding minigenes described above. The transfected or transduced cells are then returned to the subject. While such cells would preferably be lymphoid cells, they could also be any of a wide range of types including, without limitation, fibroblasts, bone marrow cells, macrophages, monocytes, dendritic cells, epithelial cells, endothelial cells, keratinocytes, or muscle cells in which they act as a source of the APL for as long as they survive in the subject. The use of lymphoid cells would be particularly advantageous in that such cells would be expected to home to lymphoid tissue (e.g., lymph nodes or spleen), and thus the APL would be produced in high concentration at the site where they exert their effect, i.e., activation of an immune response. By using this approach, active in vivo immunization with the APL is achieved. The same genetic constructs and trafficking sequences described for the in vivo approach can be used for this ex vivo strategy.

The ex vivo methods include the steps of harvesting cells from a subject, culturing the cells, transducing them with an expression vector, and maintaining the cells under conditions suitable for expression of the APL. These methods are known in the art of molecular biology. The transduction step is accomplished by any standard means used for ex vivo gene therapy, including calcium phosphate, lipofection, electroporation, viral infection, and biolistic gene transfer. Alternatively, liposomes or polymeric microparticles can be used. Cells that have been successfully transduced are then selected, for example, for expression of the minigene or of a drug resistance gene. The cells may then be lethally irradiated (if desired) and injected or implanted into the patient.

These methods of the invention can be applied to any of the diseases and species listed here. Methods to test whether an APL is therapeutic for or prophylactic against a particular disease can be simple modifications of the above-described methods for establishing the type of CD4⁺ T lymphocyte response elicited by a particular APL. Where a therapeutic effect is being tested, a test population displaying symptoms of the disease (e.g., cancer patients) is treated with a test APL, using any of the above described strategies. A control population, also displaying symptoms of the disease, is treated, using the same methodology, with a placebo. Disappearance or a decrease of the disease symptoms in the test subject would indicate that the APL was an effective therapeutic agent.

By applying the same strategies to subjects prior to onset of disease symptoms (e.g., experimental animals in which an appropriate disease can be deliberately induced, e.g., tumor model), APL can be tested for efficacy as prophylactic agents, i.e., vaccines. In this situation, prevention of onset of disease symptoms is tested.

The following examples are meant to illustrate, not limit, the invention.

### EXAMPLE 1

### Identification of HLA Class II Binding CEA Peptide Epitopes

Materials and Methods

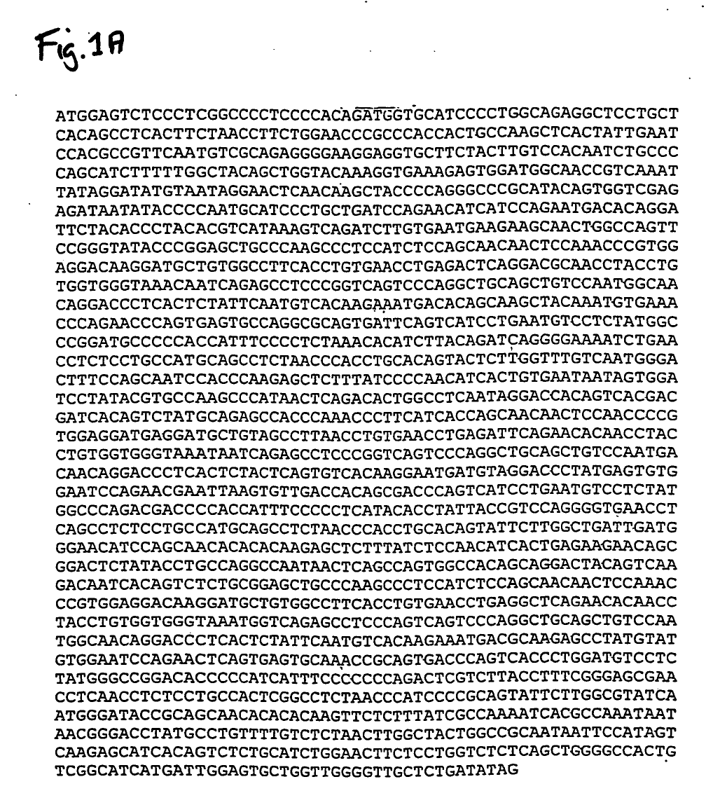

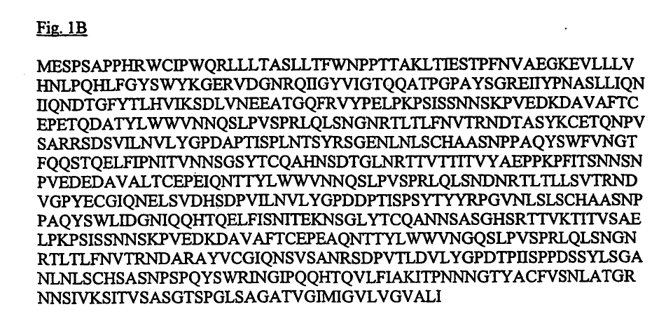

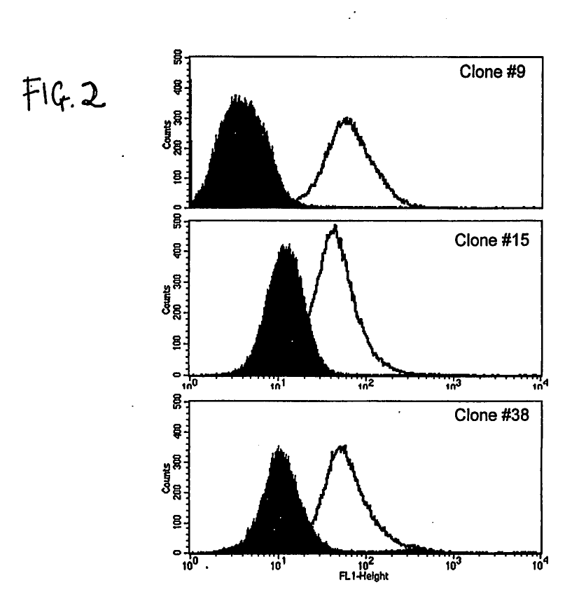

Epstein-Barr Virus (EBV)-Transformed B cell Lines expressing CEA encoding cDNA. EBV-transformed B lymphocyte lines, JY and A2.140, were propagated in RPMI 1640 medium supplemented with glutamine, penicillin/streptomycin and 10% fetal calf serum (FCS). CEA-encoding cDNA was inserted into the expression vector pcDNA3.1-Zeo (Invitrogen, Carlsbad, Calif.) at the EcoR V and Xba I restriction sites to give pcDNA3.1-CEA. pcDNA3.1-Zeo has a CMV promoter and a zeomycin selection marker. The sequence of the CEA-encoding cDNA (SEQ ID NO:12) inserted into the vector is shown in FIG. 1A and the amino acid sequence (SEQ ID NO:17) of CEA encoded by the cDNA is shown in FIG. 1B. pcDNA3.1-CEA and pcDNA3.1 (a negative control vector which was pcDNA3.1-Zeo without a protein-encoding insert) were linearized and both transfected by electroporation into JY and A2.140 cells. Stably transfected cells were selected with Zeonycin (50 μg/ml; sold as Zeocin™) and the transfected cells were cloned by limiting dilution. All surviving clones were then tested for CEA expression using the murine mAb COL-1 (specific for human CEA) by western blot and FACS analysis. Three high-expressing CEA clones were selected for further analysis (JY-CEA-9, A2.140-CEA-15 and A2.140-CEA-38). FIG. 2 shows the cell surface expression of CEA on JY-CEA-9 cells as detected by FACS analysis.

Culture of EBV-Transformed B Cell Lines. EBV-transformed B lymphocyte lines were propagated in RPMI 1640 medium supplemented with glutamine, penicillin/streptomycin and 10% fetal calf serum (FCS) in 2 L roller bottles to a density of ˜10⁶ cells/mL. JY cells and transfectants produced using them (e.g., JY-CEA-9) have the HLA-DRB*0401/HLA-DRB1*1301 genotype. A2.140 and transfectants produced using them (e.g., A2.140-CEA-15 and A2.140-CEA-38) have the HLA-DRB1*0101 genotype.

HLA class II purification. Cells were harvested and pelleted by centrifugation. The cell pellets were weighed to determine the cellular mass and then frozen at −80° C. prior to lysis. Each cell pellet was resuspended in lysis buffer (2 ml per gram of pellet) containing 1% CHAPS, 500 mM NaCl, 20 mM Tris-OH pH 8.0, in Milli-Q™ reverse osmosis quality (about 18.2 μΩ) water containing freshly added protease inhibitors (100 μM iodoacetamide, 8 μg/ml aprotinin, 10 μg/mL leupeptin, 10 μg/mL pepstatin A, 5 mM EDTA, 0.04% sodium azide, 1 mM PMSF), and the cells were lysed by gentle agitation for 1 hr at 4° C. on a rotor table. The resulting cell lysate was sedimented by ultra-centrifuge for 1 hr at 230,000×g at 4° C. (37,500 RPM on a SW41 TI rotor). The insoluble material was removed by sedimentation at 175,000×g for 2 hours, and the soluble supernatant fraction used for subsequent HLA purification. Multi-modal protein purification using HPLC columns was achieved by coupling the chromatographic sorbents in series with automated switching valves that direct the class II HLA-peptide complex containing effluent to subsequent columns in the sequences. The first three coupled columns were connected directly in series and acted together as a single pre-clearing column using high strength large throughpore perfusion sorbents (6000-8000 Å throughpores and 500-1000 Å diffusive pores, 50 μm) coated and crosslinked with a hydrophilic stationary phase and covalently conjugated with Protein A as the sorbent. These columns were designed to remove those proteins that non-specifically bind to the sorbents. Column 1 contained unmodified Protein A sorbent, column 2 contained Protein A coated with normal mouse serum, and column 3 was Protein A coated with bovine serum. The pre-clearing columns were followed by an immunoaffinity column of Protein A coupled with mAb specific for a non-polymorphic determinant on HLA-DR molecules (A-LB3.1). After passing the lysate through the immunoaffinity column, the column was extensively washed with 50 column volumes of 0.1% CHAPS/500 mM NaCl/0.05% sodium azide/20 mM Tris-OH pH 8.0, followed by 50 column volumes of 0.1% DOC/20 mM Mops/280 mM NaCl/0.05% sodium azide pH 8.0, and finally 100 column volumes of 0.1% DOC/0.05% sodium azide/10 mM Tris-OH pH 8.0. The HLA-DR-peptide complexes were eluted from the immunoaffinity support using 3.5 column volumes of 50 mM carbonate/0.1% DOC/0.05% NaN₃ at pH 11.5.

Peptide Analysis

The HLA class II protein samples can be concentrated by various methods. In one such method, the HLA class II protein sample eluted from the immunoactivity column was concentrated to 100 μl using an ultrafiltration device (Amicon Centricon 10) prior to peptide extraction. Naturally processed peptide mixtures were acid eluted from HLA class II molecules by adding 800 μl 10% acetic acid and incubating for 15 minutes at 70° C., as previously described [Chicz et al. (1993), J. Exp. Med. 178:24-47]. The peptides were separated from the remaining HLA protein by ultrafiltration with an Amicon Centricon 10™ device. The “flow-through” fraction containing the acid-extracted peptides was concentrated on a Savant SpeedVac™ centrifugal vacuum concentrator to a volume of approximately 20-30 μl and stored at −80° C. The acid-extracted peptide mixtures were then separated by reverse phase chromatography as previously described [Chicz et al. (1993), supra] but with minor modifications. Briefly, peptide solutions were preconcentrated by trapping the peptides using a small bed (0.5-3.0 μL bed volume) of polymeric reversed phase support. This also facilitates removal of hydrophilic contaminants that may be in the sample by washing the trap with a suitable aqueous solution (e.g., the buffers used for the chromatographic separation of the isolated peptides). Subsequently, peptides were back-flushed from the trapping phase to a microbore C18 column (1.0×250 mm; Vydac, Hesperia, Calif.), and peptide fractionation was performed at a flow rate of 50 μl/minute. The column effluent was collected and stored at −20° C. prior to analysis by mass spectrometry.

An alternative approach was to concentrate HLA class II molecule samples and extract the associated peptides using solid phase extraction (SPE). In this method, the HLA class II molecule samples were concentrated using a protein capture column that contained a suitable HPLC stationary phase. Appropriate HPLC stationary phases include a reversed phase (preferably a C18 or a polymeric C18-like reversed phase). However, a cation or anion exchange resin (including both strong, weak and mixed bed ion exchange phases) or other substrates that exhibit high affinity for protein complexes while enabling the elution of the HLA class II peptides, are equally suitable. Suitable dimensions of the protein capture column are 1-10 mm internal diameter and 2-10 cm long. Once the protein was bound to the capture column, the column was washed with suitable solvents (e.g., an aqueous buffer containing 5-25 mM Tris base at pH 7.5-8.5 followed by an aqueous solution of trifluroacetic acid (0.05-0.2% v/v)) to remove hydrophilic contaminants. The HLA class II molecule-peptide complexes were next disrupted by the action of a suitable solvent that also facilitated the elution of peptides from the protein capture column. An appropriate solvent for this purpose is a mixture of acetonitrile (5-25% v/v), TFA (0.05-5% v/v) in water. The eluted peptides were collected in a suitable container (e.g., an Eppendor™ microfuge tube) and concentrated using a Savant Speedvac™ centrifugal vacuum concentrator. This step lowered the acetonitrile concentration and thereby permitted peptide fractionation by reverse phase chromatography (as described above). All peptide samples were stored at −20° C. prior to analysis by mass spectrometry. Alternatively, the eluted peptides were mixed post-elution from the protein capture column with an aqueous solution of TFA (typically 0.05-0.2% TFA in water) in order to lower the acetonitrile concentration and thereby permit peptide adsorption onto a peptide capture column. The peptide capture column contained an HPLC phase (e.g., a reversed phase resin such as C18 or polymeric equivalent and an ion exchange resin or other suitable phase that exhibits high affinity towards peptides). Suitable dimensions of the peptide capture column are 0.5-4.6 mm internal diameter and 1-10 cm long. Following the adsorption of peptides, the peptide capture column was washed with a suitable solvent (e.g., 0-5% acetonitrile, 0.05-0.2% TFA in water) prior to peptide fractionation by reversed phase chromatography (as described above). All peptide samples were stored at −20° C. prior to analysis by mass spectrometry.

Peptide samples were prepared for matrix assisted laser desorption time-of-flight (MALDI-TOF) mass spectrometry analysis by adding 1.0 μL of each sample to a spot on the sample plate. A matrix solution (0.5 μL of a solution of α-cyano-4-hydroxycinnamic acid (10 mg/ml) in 50% acetonitrile/0.1% trifluoroacetic acid) was added to the dry sample spots and allowed to air dry. The matrix solution also contained des-arg bradykinin and glu-fibrinopeptide B (300 fmol/μL of each) of each as internal mass scale calibrants. Mass spectra were obtained at optimum laser intensities by averaging the ion signals from 50 to 256 individual scans in both linear and reflector modes using a single stage extended length reflector time-of-flight mass spectrometer (Voyager Elite XL; PerSeptive Biosystems, Framingham, Mass.).

An automated microcapillary liquid chromatography-mass spectroscopy (LC-MS) approach with either targeted or data dependent collision-assisted dissociation (CAD) was used to sequence low levels of naturally processed HLA associated peptides that were isolated by the MALDI-TOF-MS approach previously described. Peptide fractions separated by reversed phase chromatography were diluted to a final volume of 5-20 μl to aid handling and permit second dimension reversed phase separations. Each diluted peptide solution was then concentrated by trapping peptides in a small bed (0.5-1.0 μL bed volume) of polymeric reversed phase support. This step also facilitated removal of hydrophilic contaminants by washing the trap with a suitable aqueous solution (e.g., mobile phase A as used in the chromatographic separation of isolated peptides). The peptides were then back flushed from the trapping phase onto the microcapillary (with an inner diameter of 75 μm and packed with 3-10 cm of 1-7 μm 100-200 Å C₁₈ or non-porous material) and separation was developed using a non-linear gradient of conventional mobile phases for peptide separations (typically combinations of water and acetonitrile containing a suitable ion pair reagent). A mobile phase flow rate of 0.15-0.20 μL/min was achieved by splitting the flow from the pumps and using a backpressure regulator. Peptide detection was by μ-electrospray mass spectrometry. The voltage necessary to drive the electrospray was applied at the head of the microcapillary column and peptides were electrosprayed into the mass analyzer directly as they eluted from the column. CAD experiments were either predetermined to conduct specific target analyses or triggered in a data dependent mode, using ions that were more abundant than a user-set threshold. Dynamic exclusion was used in conjunction with data dependent analyses to ensure maximum peptide coverage (i.e., minor responses were analyzed by CAD following a user-determined number of CAD experiments of a single peptide response) by writing an exclusion list during assay progression so that a given ion will not be analyzed by multiple CAD experiments. The time that a given ion resides on the exclusion list was dependent upon the quality of the chromatographic separations. This time is determined experimentally. In this way, separated isobaric responses may be analyzed. Peptide sequencing sensitivities greater than 100 attomoles were achieved using this method.

Results

A cDNA sequence encoding the tumor antigen CEA (SEQ ID NO:12) was cloned into a mammalian expression vector (pcDNA3.1-Zeo) and was transfected into and expressed in JY EBV-transformed B lymphocytes and A2.140 EBV-transformed B lymphocytes using the methods described above. Transfectants were selected using Zeocin and the resulting stable transfectants were cloned by limiting dilution. Six control clones stably transfected with the pcDNA3.1-Zeo vector without an expressible insert (“parental vector”) and 43 CEA expressing transfectant clones were generated with JY cells. Six control clones stably transfected with the parental vector and 7 CEA expressing transfectant clones were generated with A2.140 cells. All clones were then tested for CEA expression by western blot and FACS analysis using the murine mAb Col-1. Three CEA high-expressing clones (one from JY cells and two from A2.140 cells) were selected for further analysis (JY-CEA-9, A2.140-CEA-15 and A2.140-CEA-38). Two control clones, one each from JY (clone JY-pcDNA3-1G9) and A2.140 cells (clone A2.140-pcDNA3) were also selected for peptide repertoire comparison.

After growing the cells to a density of ˜10⁶ cells/mL, each preparation was harvested by centrifugation. JY-pcDNA3-1G9 was harvested from 10.8 L of medium to yield 24.0 grams of cell pellet. JY-CEA-9 was harvested from 10.8 L of medium to yield 23.6 grams of cell pellet. A2.140-pcDNA3 was harvested from 20 L of medium to yield a 41.0 gram pellet. A2.140-CEA-15 was harvested from 14.4 L of medium to yield a 33.0 gram pellet. A2.140-CEA-38 was harvested from 14.4 L of medium to yield a 30.2 gram pellet.

Protein purification was accomplished by immunoaffinity chromatography as described above. HLA-DR molecules purified from JY are heterogenous and include both HLA-DR4 and HLA-DR13 molecules. The JY-pcDNA3-lG9 HLA-DR yield from the Protein-A-LB3.1 column was 5.8 mg. The JY-CEA-9 HLA-DR yield from the Protein-A-LB3.1 column was 5.0 mg. The only HLA-DR molecules A2.140 cells express are HLA-DR1 molecules. The A2.140-pcDNA3 HLA-DR1 yield from the Protein-A-LB3.1 column was 12.0 mg. The A2.140-CEA-15 HLA-DR1 yield from the Protein-A-LB3.1 column was 12.5 mg. The A2.140-CEA-38 HLA-DR1 yield from the Protein-A-LB3.1 column was 8.0 mg.

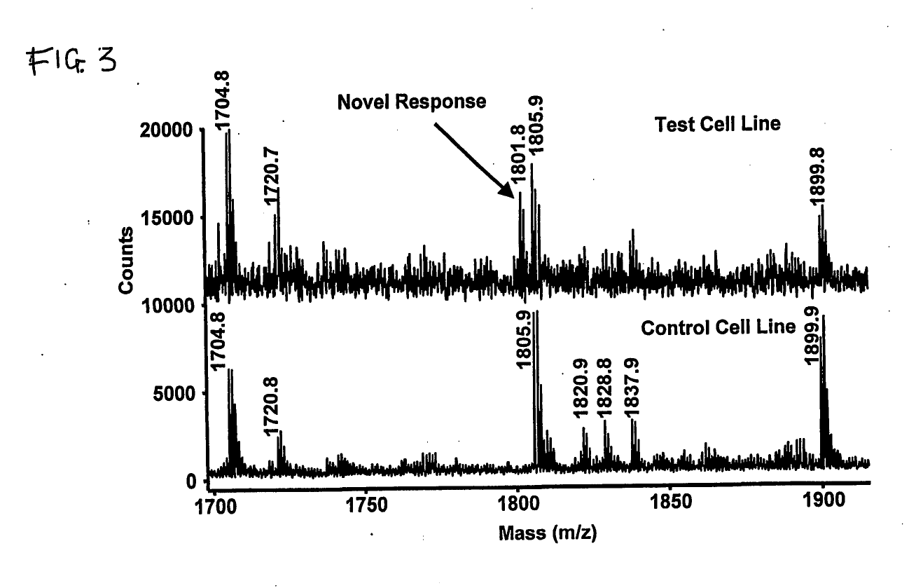

Peptides were eluted from each HLA-DR protein preparation and separated by RP-HPLC, and each of the 100 fractions collected was analyzed by MALDI-TOF. RP-HPLC analysis was highly reproducible, with chromatographic traces from the CEA-expressing and control CEA-non-expressing HLA-DR4/13 preparations showing similarity to each other. Similarly, the traces obtained with peptides from CEA-expressing and control CEA non-expressing HLA-DR1 preparations showed similarity to each other. The chromatographic traces of the peptide repertoires from HLA-DR4/13-expressing JY cells and HLA-DR1-expressing A2.140 cells were distinguishable from each other. A subtractive approach was used to identify CEA-derived peptides. Mass spectra of equivalent RP-HPLC fractions from the A2.140-pcDNA3 and A2.140-CEA-38 peptides preparations were overlaid and masses common to both were discounted from further analysis. An example of such a profile is shown in FIG. 3. In FIG. 3, while peaks with m/z values of about 1704.8, 1805.9 and 1899.9 were seen in the spectra obtained with peptide mixtures from both A2.140-CEA-38 and A2.140-pcDNA3 cells, a peak with a m/z value of 1801.9 was seen only in the spectrum obtained with the peptide mixture from A2.140-CEA-38 cells. Similar MALDI-TOF analyses were performed for the peptide repertoires isolated from JY-CEA-9 and JY-pcDNA3-1G9 control cell lines as well as A2.140-CEA-15 and A2.140-pcDNA3 cell line control.

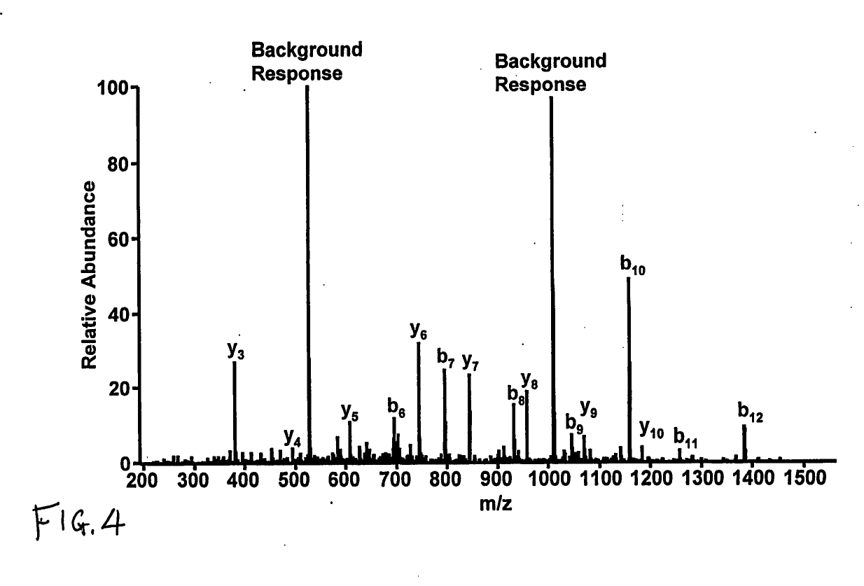

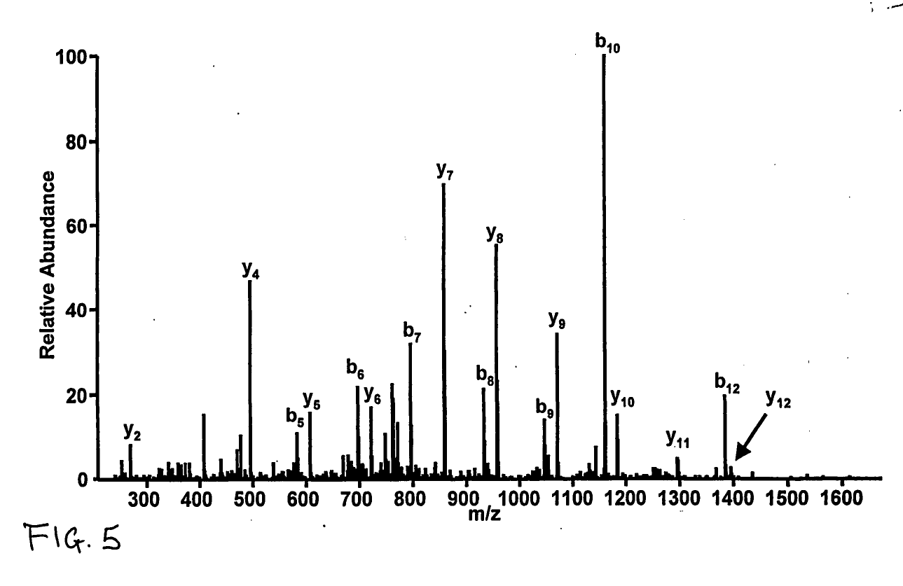

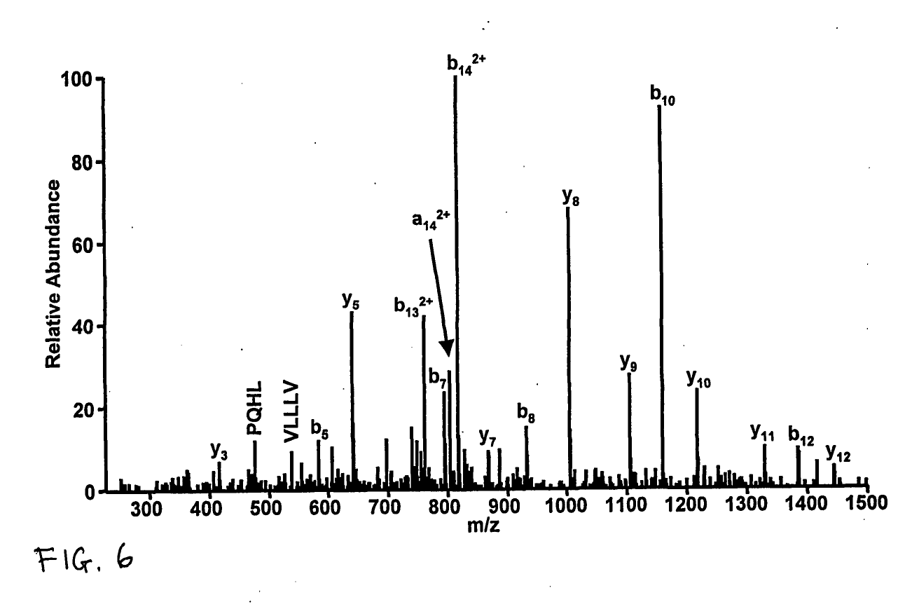

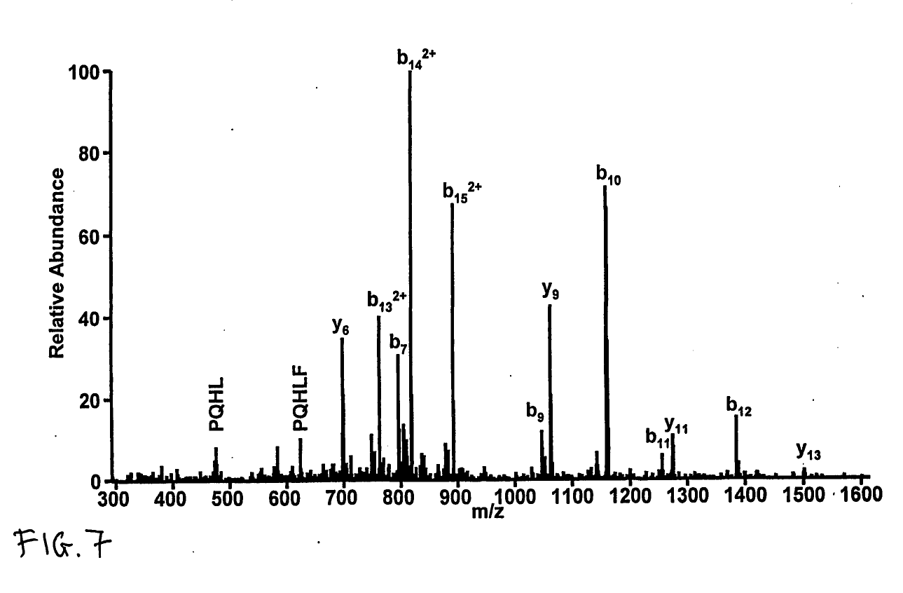

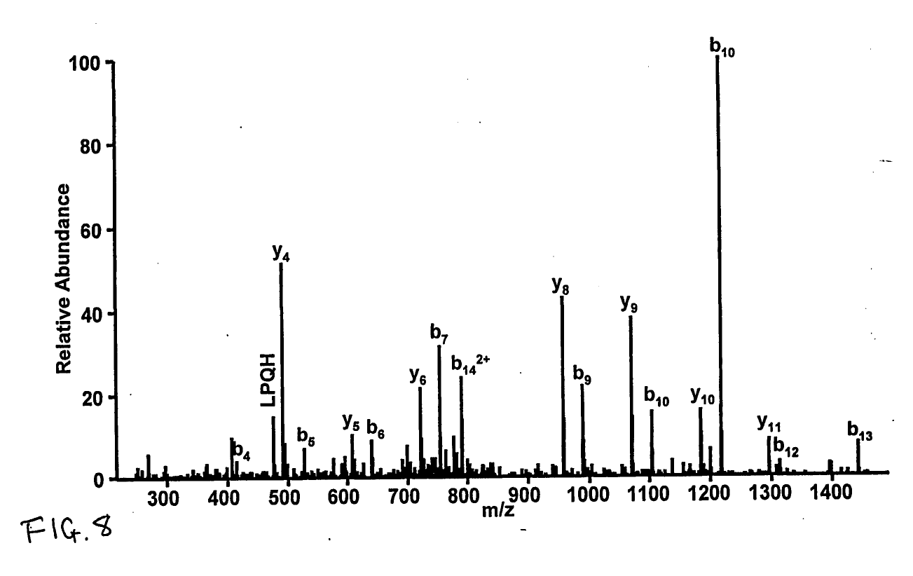

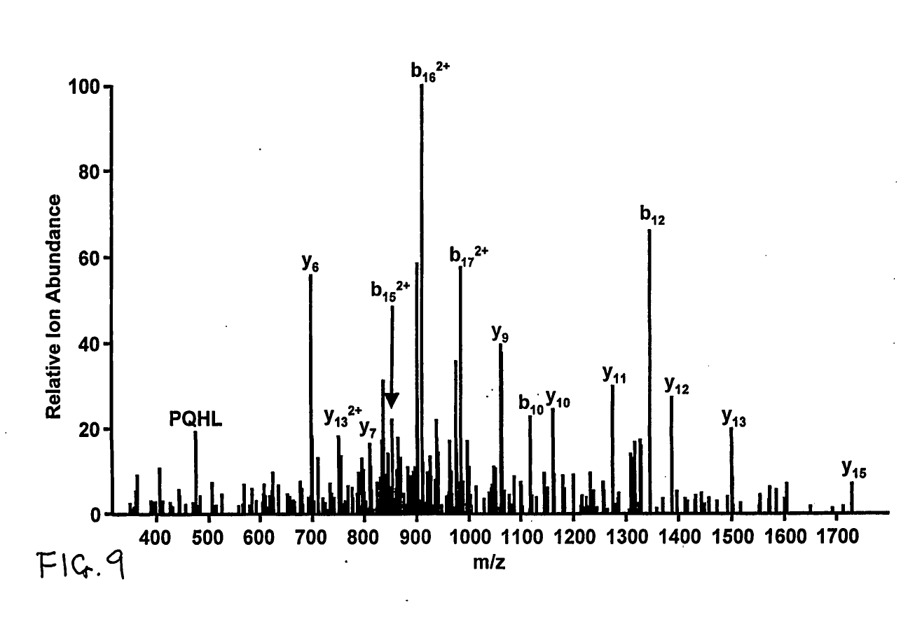

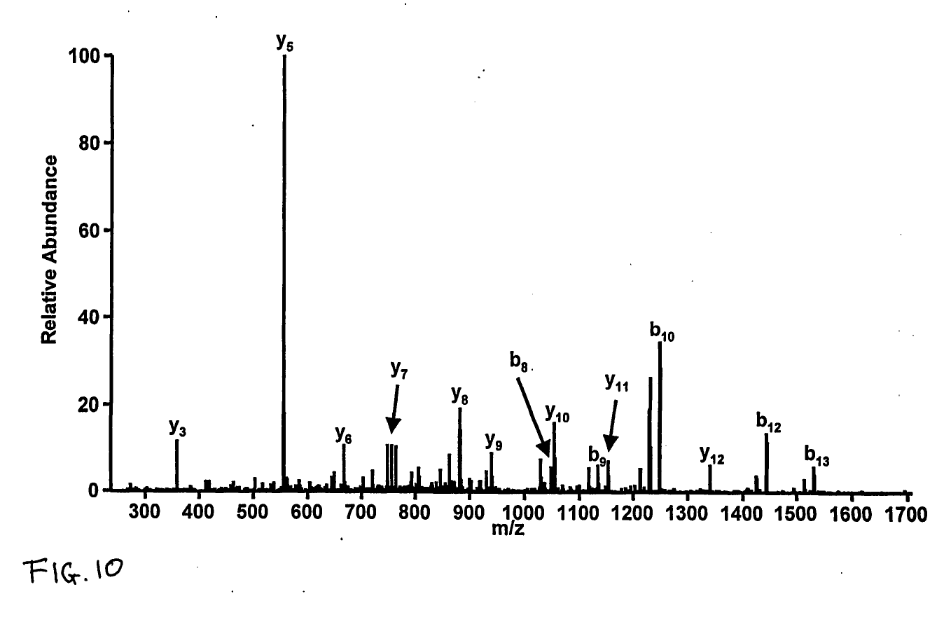

Of the approximately 40,000 m/z values observed, 165 novel masses were initially identified as potential naturally processed peptides from CEA. Subsequent mass analyses using MS/MS fragmentation were performed on 278 of the 500 RP-HPLC fractions as described. Approximately 50,000 MS/MS experiments were performed in both data dependent and targeted mass analyses. Six CEA peptides from the JY-CEA-9 cell line (SEQ ID NOS:1-6) were identified and sequenced (FIGS. 4-9). One CEA-derived peptide was found in both the A2.140-CEA-15 and A2.140-CEA-38 cell lines (SEQ ID NO:7) (FIG. 10).

Thus, the described IMF method applied to the analysis of peptides produced by natural processing of CEA identified seven peptides that are associated with HLA-DR4/13 and HLA-DR1. This knowledge provides the basis for the development of therapeutic and/or prophylactic agents against CEA associated cancers, e.g., colon cancer. It is expected that analogous methodologies can be similarly successful in identifying other class II MHC-restricted tumor antigen peptides that activate CD4⁺ T cells and are involved in the CD4⁺ T lymphocyte-mediated pathogenesis of other diseases (see above) in which susceptibility is linked to the expression of a particular class II MHC molecule.

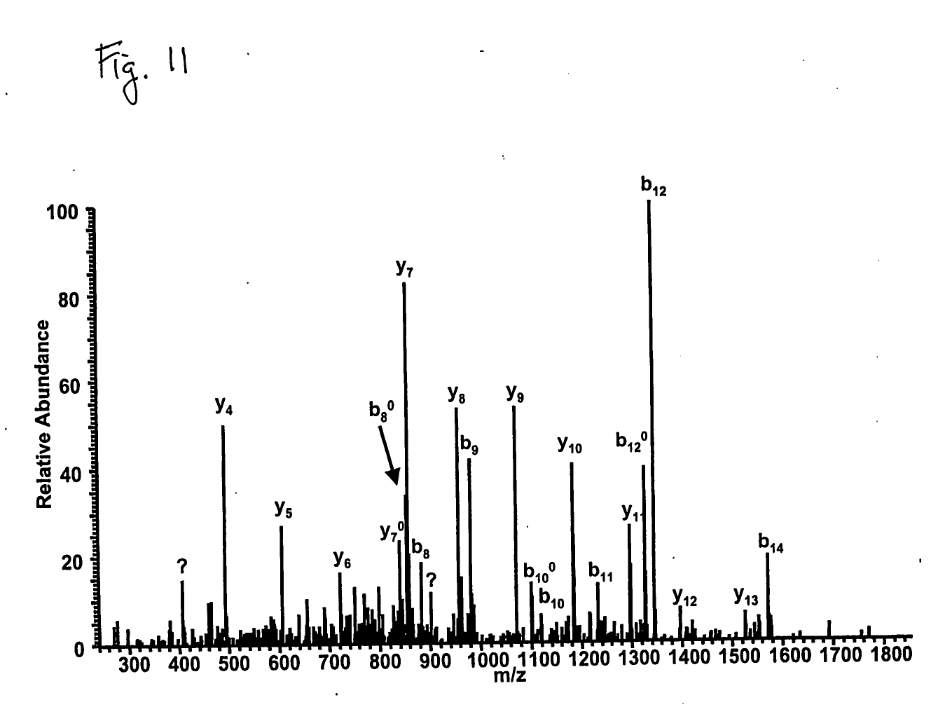

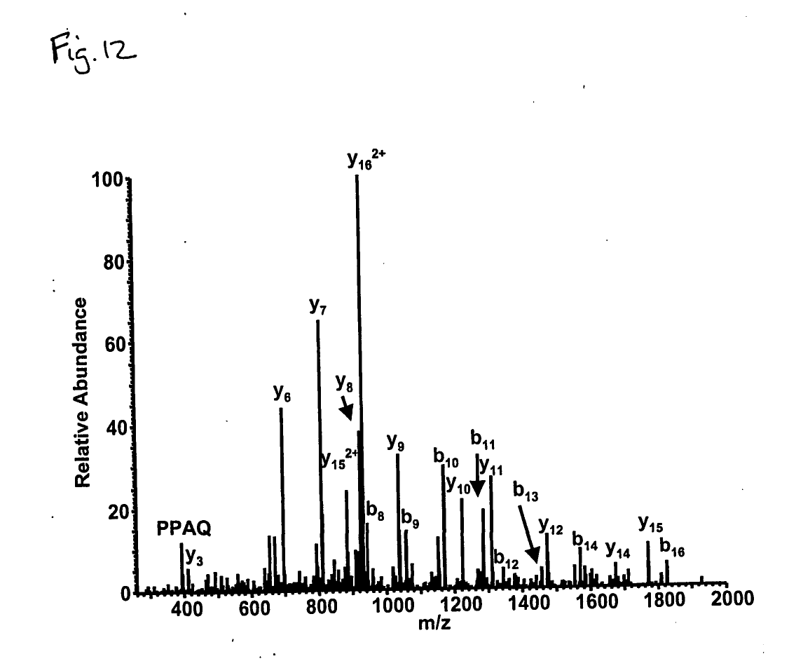

Three additional peptides (SEQ ID NOS: 13-15) from the JY-CEA-9 cell line were subsequently identified and sequenced (FIGS. 10-12).

Epitope Verification (EV):

To confirm that peptide epitopes identified are relevant to cancer (i.e., that they are recognized by CD4⁺ T cells of patients with cancer), T cell proliferation assays can be carried out using synthetic peptides having amino acid sequences based upon the sequence of the peptides identified by mass spectrometry to be derived from CEA (e.g., the peptides with SEQ ID NOS:1-7 and 13-15). Peptides can be synthesized using Fmoc chemistry and purified by RP-HPLC. The amino acid sequences and purity of greater than 90% for all the synthetic peptides can be confirmed by MALDI-MS and analytical HPLC. Peripheral blood mononuclear cells from recent onset cancer patients (and healthy controls expressing the appropriate HLA-DR1 and HLA-DR4 molecules) can be separated by density gradient centrifugation and co-cultured in wells of 96-well U-bottom plates with peptides at a concentration of 10 μg/ml for 5 days in 150 μl RPMI 1640/10% pooled normal AB serum, followed by pulsing with 0.5 μCi [³H]-thymidine/well and harvesting onto filters for radioactivity counting measured in counts per minute (cpm). There are generally twelve replicate wells per test group. Results are expressed as a stimulation index (SI) which is the ratio of the counts per minute (cpm) obtained in samples from cultures containing peptide to the cpm obtained in samples from cultures without peptide (mean cpm of 12 wells in each case). The data are also analyzed in terms of the fraction of “positive culture wells.” A positive culture well is one that contained peptide and resulted in cpm>mean cpm+2SD obtained from cultures without peptide. T cell responses are considered significant when the SI is >2.0 and >40% wells are positive.

Data obtained from the above EV analysis are expected to confirm that the peptides identified by the IMF method are recognized specifically by CD4⁺ T lymphocytes from HLA-DR1 and HLA-DR4 expressing cancer patients and thus may be implicated in immunity to the cancer.

### EXAMPLE 2

### Binding of Consensus Peptides to Isolated HLA-DR1 and HLA-DR4 Molecules

Synthetic peptides with amino acid sequences based on the 3 core regions identified by the IMF can be tested for their ability to bind to isolated HLA-DR1 and HLA-DR4 molecules in binding inhibition assays performed essentially as previously described [Chicz et al. (1997), J. Immunol. 159: 4935-4942]. In brief, aliquots of immunopurified preparations of HLA-DR1 and HLA-DR4 (final concentration of 10 μg/ml) are incubated with a biotinylated HLA-DR specific binding peptide (consisting of residues 98-117 of class II MHC invariant chain) (“the indicator peptide”) (1 μM) and varying concentrations of the test peptides in 0.2 ml tubes. After an overnight incubation at room temperature, the contents of each tube are transferred to a well of a 96-well plastic microtiter plate precoated with anti-HLA-DR antibody. The microtiter plates are rocked for 60 min at room temperature and unbound material is removed by rigorous washing. The relative amount of bound standard peptide in each well will be determined by measuring color development after addition of streptavidin-conjugated alkaline phosphatase, washing, and adding a chromogenic alkaline phosphatase substrate.

Other Embodiments

The invention also features the following embodiments.

Methods of Use

The peptides or APL of the invention can be used to activate CD4⁺ memory T cells specific for CEA by in vitro methods known to those in the art. After a patient undergoes surgery to remove a tumor, such CEA-specific CD4+memory T cells may be administered to establish tumor-specific, long-lasting immunity against tumor rechallenge. Topalian [Current Opinion in Immunology (1994), 6:741-745] reviews the role of anti-tumor CD4⁺ T cells in immunological memory. Studies have demonstrated that tumor-specific immunological memory can be established by vaccination with IL-2-transduced tumor cells, and CD4⁺ T cells are a critical component of this memory response.

The peptides or APL of the invention can also be used to help class I MHC restricted (CD8⁺) CTL responses. For example, CD8⁺ T cells recognizing the gag-L-encoded CTL epitope have been demonstrated to be effector cells that are efficiently activated with help from peptide-activated, tumor-specific CD4⁺ T cells [Ossendorp et al. (1998), J. Exp. Med. 187:693-702]. In addition, mature, monocyte-derived dendritic cells were used to elicit resistance to malignant melanoma. [Thurner et al. (1999), J. Exp. Med. 190:1669-1678]. Intracutaneous injections of Mage-3A1 peptide-pulsed mature dendritic cells enchanced Mage-3A1-specific CD8+CTL responses and activated CD4⁺ T cell activity in stage 1V melanoma patients with large tumor loads.

CD4⁺ T cells have been shown to play a role in the induction and persistence of CD8+T cells. A peptide epitope h-gp100 (melanocyte differentiation antigen which is expressed by more than 75% of melanomas) that binds to several class II MHC molecules was identified and characterized. The MHC class II-restricted epitope, which was capable of inducing tumor-reactive CD4⁺ cells, could potentiate antitumor effector function and long term immunity [Touloukian et al. (2000), J. Immunol. 164:3535-3542]. The peptide was able to induce specifically the in vitro expansion of CD4⁺ T cells. Thus, the peptides or APL of the invention could be used as immunogens, together with one or more CD8⁺ T cell epitopes produced by appropriate CEA-expressing tumor cells. Interestingly in this regard, the human Melan-A/MART-1 protein that includes an HLA-A2-restricted peptide epitope recognized by melanoma-reactive CD8⁺ CTLs also contains at least one HLA-DR4-presented peptide recognized by CD4⁺ T cells [Zarour et al. (2000), Proc. Natl. Acad. Sci. USA 97:400-405].

The peptides or APL of the invention can also be used to activate cytokine production for the recruitment of tumoricidal eosinophils and macrophages. Hung et al. [(1998), J. Exp. Med. 188:2357-2368] showed that vaccination with irradiated tumor cells transduced to secrete granulocyte/macrophage colony-stimulating factor resulted in activation of CD4⁺ T cells which, by the action of the cytokines that they produce, activated eosinophils and macrophages to produce superoxide and nitric oxide, both which have tumoricidal activity. It will be possible to screen for a peptide or APL of the invention that will stimulate CD4⁺ T cells with such activity.

Qin et al. [(2000), Immunity 12:677-686] showed that in vivo CD4⁺ T cell mediated immunity to tumors was due to inhibition of tumor-induced angiogenesis, was dependent on IFN-γ, and required expression of the receptor for IFN-γ on non-hematopoietic cells. Peptides or APL of the invention can be useful in activating CD4⁺ T cells with this anti-tumor activity.

Reagents that Bind to Peptide-Class II MHC Complexes

Reagents that bind to peptide-class II MHC complexes can be made, for example, by screening a phage display library in which the phage particles contain nucleic acid sequences encoding antibody fragments such as Fab or single chain Fv (scFv) fragments. Such libraries can be screened by testing for the presence of and isolating phage particles with the ability to bind to the peptides of the invention (e.g., KEVLLLVHNLPQH (SEQ ID NO:1), KEVLLLVHNLPQHL (SEQ ID NO:2); KEVLLLVHNLPQHLF (SEQ ID NO:3); KEVLLLVHNLPQHLFG (SEQ ID NO:4); GKEVLLLVHNLPQHL (SEQ ID NO:5); EGKEVLLLVHNLPQHLFG (SEQ ID NO:6); YLWWVNGQSLPVSPR (SEQ ID NO:7); EGKEVLLLVHNLPQHL (SEQ ID NO:13); NPPAQYSWLIDGNIQQH (SEQ ID NO:14); or NPPAQYSWLIDGNIQQHT (SEQ ID NO:15)), bound to a class II MHC molecule of interest. For example, phage display technology has been used to identify antibodies specific for defined HLA-DR2 peptide complexes associated with multiple sclerosis [Krogsfaard et al. (2000), J. Exp. Med. 191:1395-1412]. Alternatively, polyclonal antibodies or mAb can be screened for their ability to bind to peptide-class II MHC complexes of interest.

The above antibody-based reagents can be used, for example, in diagnosing cancer. For example, binding to a test cell of a Fab, scFv, or an antibody (e.g., a mAb) specific for a HLA-DR4 molecule bound to a CEA peptide of the invention would indicate that the test cell is a cancer cell. Examples of detection agents used to detect binding of antibodies or antibody fragments include, without limitation, enzymes, radiolabels, luminescent compounds, and fluorescing compounds that elicit a detectable and measurable signal when the antibody complexes with the CEA naturally processed peptide. Examples of detectors include, without limitation, spectrophotometers, calorimeters, fluorometers, luminometers and biacore machines.

Therapeutic agents for treating cancer can be made, for example, by linking one of the above reagents with a therapeutic (e.g., cytotoxic) atom or molecule. An appropriate therapeutic agent, after administration (by any of the methods disclosed herein) to a subject with the relevant cancer, binds to the cancer cells. The therapeutic atom or molecule can then kill the cancer cell. Alternatively, the therapeutic agent is internalized by the cancer cell and then the therapeutic atom or molecule kills the cancer cell. The linkage between the reagent and therapeutic atom or molecule can be a covalent one or a relatively weak non-covalent one such that the complex dissociates after binding to the cancer cell surface. Examples of therapeutic atoms and molecules include chemotherapeutic compounds, radioisotopes, and toxins, e.g., ricin or diptheria toxin, or toxic fragments of such toxins.

Although the invention has been described with reference to the presently preferred embodiments, it should be understood that various modifications can be made without departing from the spirit of the invention. Accordingly, the invention is limited only by the following claims.

What is claimed is:

1. A method of identifying a class II MHC-binding fragment of a polypeptide, the method comprising:
(a) providing a mammalian antigen-presenting cell (APC) cell line transfected with a DNA encoding a polypeptide;
(b) isolating from the APC a class II MHC molecule bound to a peptide, wherein the peptide is a class II MHC-binding fragment of the polypeptide;
(c) eluting the peptide from the class II MHC molecule; and
(d) identifying the peptide as a fragment of the polypeptide.

2. The method of claim 1, wherein the polypeptide has the sequence of a tumor antigen.

3. The method of claim 1, wherein the APC is a dendritic cell.

4. The method of claim 1, wherein the APC is a macrophage or a monocyte.

5. The method of claim 1, wherein the APC is a B lymphocyte.

6. The method of claim 1, wherein the mammal is a human.

7. The method of claim 2, wherein the class II MHC molecule is selected from the group consisting of a HLA-DR molecule, a HLA-DQ molecule, and a HLA-DP molecule.

8. The method of claim 2, wherein the class II MHC molecule is a DR molecule with a β-chain encoded by a gene selected from the group consisting of DRB1*0401, DRB1*0101, DRB1*0301, DRB1*0701, DRB1*1101, DRB1*1301, DRB1*1302, DRB1*1501 and DRB5*0101.

9. An isolated peptide fewer than 35 amino acid residues in length, comprising a sequence KEVLLLVHNLPQH (SEQ ID NO:1).

10. The isolated peptide of claim 9, comprising an amino acid sequence selected from the group consisting of:
KEVLLLVHNLPQHL (SEQ ID NO:2); KEVLLLVHNLPQHLF (SEQ ID NO:3); KEVLLLVHNLPQHLFG (SEQ ID NO:4); GKEVLLLVHNLPQHL (SEQ ID NO:5); EGKEVLLLVHNLPQHLFG (SEQ ID NO:6); and EGKEVLLLVHNLPQHL (SEQ ID NO:13).

11. An isolated peptide fewer than 35 amino acid residues in length, comprising a sequence YLWWVNGQSLPVSPR (SEQ ID NO:7).

12. An altered peptide ligand (APL), the amino acid sequence of which is identical, except for 1-6 amino acid substitutions, to a fragment of carcinoembryonic antigen (CEA), the fragment being fewer than 35 amino acids residues in length and comprising a sequence selected from the group consisting of KEVLLLVHNLPQH (SEQ ID NO:1), YLWWVNGQSLPVSPR (SEQ ID NO:7), and NPPAQYSWLIDGNIQQH (SEQ ID NO:14), wherein no more than 30% of the amino acid residues of the fragment are substituted with different amino acid residues in the APL, and wherein the APL binds to a class II MHC molecule.

13. The APL of claim 12, wherein the sequence of the fragment is selected from the group consisting of KEVLLLVHNLPQH (SEQ ID NO:1), YLWWVNGQSLPVSPR (SEQ ID NO:7), and NPPAQYSWLIDGNIQQH (SEQ ID NO:14).

14. A process for making an APL, the process comprising:
(a) carrying out the method of claim 1, and
(b) synthesizing an APL consisting of a sequence which is identical to that of the peptide, except having amino acid substitutions at 1, 2, 3, 4, 5, or 6 positions in the peptide.

15. The process of claim 14, wherein the polypeptide is CEA.

16. A method of activating T cell reactivity in a mammal, the method comprising:
(a) providing (i) a peptide, the sequence of which consists of the sequence of a naturally processed fragment of CEA, wherein the peptide binds to a class II MHC molecule of the mammal and elicits a CD4⁺ T cell response, or (ii) a DNA encoding a polypeptide selected from the group consisting of (1) the peptide, (2) the peptide plus an amino terminal methionine residue, and (3) either (1) or (2) linked to a trafficking sequence; and
(b) administering the peptide or DNA to the mammal.

17. The method of claim 16, wherein the peptide is fewer than 35 amino acid residues in length and comprises a sequence KEVLLLVHNLPQH (SEQ ID NO:1).

18. The method of claim 17, wherein the peptide is KEVLLLVHNLPQH (SEQ ID NO:1).

19. The method of claim 17, wherein the peptide is KEVLLLVHNLPQHL (SEQ ID NO:2).

20. The method of claim 17, wherein the peptide is KEVLLLVHNLPQHLF (SEQ ID NO:3).

21. The method of claim 17, wherein the peptide is KEVLLLVHNLPQHLFG (SEQ ID NO:4).

22 The method of claim 17, wherein the peptide is GKEVLLLVHNLPQHL (SEQ ID NO:5).

23. The method of claim 17, wherein the peptide is EGKEVLLLVHNLPQHLFG (SEQ ID NO:6).

24. The method of claim 16, wherein the peptide is fewer than 35 amino acid residues in length and comprises a sequence YLWWVNGQSLPVSPR (SEQ ID NO:7).

25. The method of claim 24, wherein the peptide is YLWWVNGQSLPVSPR (SEQ ID NO:7).

26. A method of altering a T cell response in a mammal, the method comprising:
(a) providing (i) an APL having a sequence identical, except for amino acid substitutions at 1-6 positions, to the sequence of a naturally-processed fragment of CEA, wherein the APL binds to a class II MHC molecule of the mammal, or (ii) a DNA encoding a polypeptide selected from the group consisting of (1) the APL, (2) the APL plus an amino terminal methionine residue, and (3) either (1) or (2) linked to a trafficking sequence; and
(b) administering the APL or DNA to the mammal.

27. The method of claim 1, further comprising:
(e) providing CD4⁺ lymphocytes from a mammal having a condition suspected of being associated with presentation of the peptide by the class II MHC molecule, wherein the APCs of the mammal bear the class II MHC molecule;
(f) providing a population of APCs that bear the class II MHC molecule with the peptide bound thereto;
(g) contacting the population of APCs of (f) with the CD4⁺ lymphocytes of (e); and
(h) determining whether the CD4⁺ lymphocytes recognize the class II MHC-bound peptide, as an indication that presentation of the peptide to CD4⁺ T lymphocytes is associated with the condition.

28. The method of claim 27, wherein said presentation is associated with a pathological response of CD4⁺ T lymphocytes.

29. The method of claim 27, wherein said presentation is associated with a protective response of CD4⁺ T lymphocytes.

30. A method of diagnosis comprising:
(a) providing a CD4⁺ lymphocyte from an individual suspected of having or being susceptible to cancer;
(b) providing an APC which bears on its surface a class II MHC molecule of an allele identical to one expressed by said individual, wherein the class II MHC molecule is bound to a CEA peptide;
(c) contacting the APC with the CD4⁺ lymphocyte; and
(d) determining whether the CD4⁺ lymphocyte recognizes the class II MHC-bound peptide, as an indication that the individual has or is susceptible to cancer,
wherein the peptide comprises an amino acid sequence selected from the group consisting of: KEVLLLVHNLPQH (SEQ ID NO:1); KEVLLLVHNLPQHL (SEQ ID NO:2;) KEVLLLVHNLPQHLF (SEQ ID NO:3); KEVLLLVHNLPQHLFG (SEQ ID NO:4); GKEVLLLVHNLPQHL (SEQ ID NO:5); EGKEVLLLVHNLPQHLFG (SEQ ID NO:6); YLWWVNGQSLPVSPR (SEQ ID NO:7); EGKEVLLLVHNLPQHL (SEQ ID NO:13); NPPAQYSWLIDGNIQQH (SEQ ID NO:14); and NPPAQYSWLIDGNIQQHT (SEQ ID NO:15).

31. A method of treating a subject suspected of having or being susceptible to cancer, the method comprising administering the peptide of claim 9 to the subject.

32. A method of identifying a reagent for diagnosing cancer, the method comprising:
(a) providing a test reagent selected from the group consisting of a Fab fragment, a monoclonal antibody (mAb), and a single chain Fv (scFv) fragment;
(b) providing a complex comprising a class II MHC molecule bound to a peptide comprising a sequence selected from the group consisting of KEVLLLVHNLPQH(SEQ ID NO:1), KEVLLLVHNLPQHL (SEQ ID NO:2); KEVLLLVHNLPQHLF (SEQ ID NO:3); KEVLLLVHNLPQHLFG (SEQ ID NO:4); GKEVLLLVHNLPQHL (SEQ ID NO:5); EGKEVLLLVHNLPQHLFG (SEQ ID NO:6); YLWWVNGQSLPVSPR (SEQ ID NO:7); EGKEVLLLVHNLPQHL (SEQ ID NO:13); NPPAQYSWLIDGNIQQH (SEQ ID NO:14); and NPPAQYSWLIDGNIQQHT (SEQ ID NO:15); and
(c) testing whether the test reagent binds to the complex, wherein binding to the complex is an indication that the test reagent is potentially useful for diagnosing cancer.

33. The method of claim 30, wherein the class II MHC molecule is a DR molecule with a β-chain encoded by a DRB1*0401 gene.

34. The method of claim 30, wherein the class II MHC molecule is a DR molecule with a β-chain encoded by a DRB1*0101 gene.

35. A method of diagnosis, the method comprising:
(a) providing a test cell from a mammalian subject;
(b) providing a reagent that binds to a CEA peptide fragment bound to a class II MHC molecule;
(c) contacting the test cell with the reagent; and
(d) detecting binding of the reagent to the test cell as an indication that the test cell is a cancer cell.

36. A method of cancer therapy comprising:
(a) providing a composition comprising a reagent selected from the group consisting of a Fab fragment, a mAb, and a scFv fragment, wherein the reagent recognizes a naturally processed CEA peptide bound to a MHC class II molecule, the reagent being linked to an agent selected from the group consisting of a chemotherapeutic compound, a radioactive isotope and a toxin; and
(b) administering the composition to a subject suspected of having or being susceptible to a cancer characterized by expression of CEA.

37. A method of identifying a class II MHC-binding fragment of a tumor antigen, the method comprising:
(a) providing a mammalian APC comprising a class II MHC molecule and the tumor antigen;
(b) isolating from the APC the class II MHC molecule bound to a peptide, wherein the peptide is a class II MHC binding fragment of the tumor antigen;
(c) eluting the peptide from the class II MHC molecule; and
(d) identifying the amino acid sequence of the peptide.

38. The method of claim 37, further comprising:
(e) providing CD4⁺ lymphocytes from a mammal having a cancer suspected of being associated with presentation of the peptide by the class II MHC molecule, wherein the APCs of the mammal bear the class II MHC molecule;
(f) providing a population of APCs that bear the class II MHC molecule with the peptide bound thereto;
(g) contacting the population of APCs of (f) with the CD4⁺ lymphocytes of (e); and
(h) determining whether the CD4⁺ lymphocytes recognize the class II MHC bound peptide, as an indication that presentation of the peptide to CD4⁺ lymphocytes is associated with the cancer.

39. An isolated DNA comprising a nucleotide sequence encoding a peptide fewer than 35 amino acids in length and comprising the sequence of SEQ ID NO:1, 7 or 14.

40. A vector comprising the DNA of claim 39.

41. The vector of claim 40, wherein the nucleotide sequence is operatively linked to a transcriptional regulatory element.

42. A cell comprising the vector of claim 40.

43. A cell comprising the vector of claim 41.

44. An isolated peptide fewer than 35 amino acid residues in length, comprising a sequence NPPAQYSWLIDGNIQQH (SEQ ID NO:14).

45. The isolated peptide of claim 44, comprising a sequence NPPAQYSWLIDGNIQQHT (SEQ ID NO:15).

46. The method of claim 17, wherein the peptide is EGKEVLLLVHNLPQHL (SEQ ID NO:13).

47. The method of claim 16, wherein the peptide is fewer than 35 amino acid residues in length and comprises a sequence NPPAQYSWLIDGNIQQH (SEQ ID NO:14).

48. The method of claim 47, wherein the peptide is NPPAQYSWLIDGNIQQH (SEQ ID NO:14).

49. The method of claim 47, wherein the peptide is NPPAQYSWLIDGNIQQHT (SEQ ID NO:15).

50. The method of claim 2, wherein the tumor antigen is CEA.

51. The method of claim 2, wherein the tumor antigen is selected from the group consisting of prostate specific antigen, MAGE 1-4, MAGE 6, MAGE 12, a mucin, tyrosinase, MART, Pmel 17, N-acetylglucoaminyltransferase V intron V sequence, Prostate Ca psm, PRAME, P-catenin, MUM-1-B, GAGE-1, BAGE 2-10, c-ERB2, Epstein-Barr Virus nuclear antigen 1-6, gp75, human papilloma virus E6 and E7, p53, lung resistance protein, Bcl-2, and Ki-67.

52. The method of claim 16, wherein the mammal is suspected of having or being susceptible to cancer.

53. An isolated peptide comprising a fragment of CEA comprising KEVLLLVHNLPQH (SEQ ID NO:1) and a further 1-15 residues of CEA sequence at the C-terminus of SEQ ID NO:1, at the N-terminus of SEQ ID NO:1, or at both the N-terminus and the C-terminus of SEQ ID NO:1.

54. An isolated peptide comprising a fragment of CEA comprising YLWWVNGQSLPVSPR (SEQ ID NO:7) and a further 1-15 residues of CEA sequence at the C-terminus of SEQ ID NO:7, at the N-terminus of SEQ ID NO:7, or at both the N-terminus and the C-terminus of SEQ ID NO:7.

55. An isolated peptide comprising a fragment of CEA comprising NPPAQYSWLIDGNIQQH (SEQ ID NO:14) and a further 1-15 residues of CEA sequence at the C-terminus of SEQ ID NO:14, at the N-terminus of SEQ ID NO:14, or at both the N-terminus and the C-terminus of SEQ ID NO:14.

56. A method of activating T cell reactivity in a mammal, the method comprising:
(a) providing (i) the peptide of claim 53, or (ii) a DNA encoding a polypeptide selected from the group consisting of (1) the peptide, (2) the peptide plus an amino terminal methionine residue, and (3) either (1) or (2) linked to a trafficking sequence; and
(b) administering the peptide or DNA to the mammal.

57. A method of activating T cell reactivity in a mammal, the method comprising:
(a) providing (i) the peptide of claim 54, or (ii) a DNA encoding a polypeptide selected from the group consisting of (1) the peptide, (2) the peptide plus an amino terminal methionine residue, and (3) either (1) or (2) linked to a trafficking sequence; and
(b) administering the peptide or DNA to the mammal.

58. A method of activating T cell reactivity in a mammal, the method comprising:
(a) providing (i) the peptide of claim 55, or (ii) a DNA encoding a polypeptide selected from the group consisting of (1) the peptide, (2) the peptide plus an amino terminal methionine residue, and (3) either (1) or (2) linked to a trafficking sequence; and
(b) administering the peptide or DNA to the mammal.

59. An isolated fragment of CEA shorter than full-length CEA, wherein the fragment comprises one or more amino acid sequences selected from the group consisting of SEQ ID NO:1, SEQ ID NO:7, and SEQ ID NO:14.

60. A method of activating T cell responsiveness in a mammal, the method comprising:
(a) providing (i) the fragment of claim 59, or (ii) a DNA encoding a polypeptide selected from the group consisting of (1) the fragment, (2) the fragment plus an amino terminal methionine residue, and (3) either (1) or (2) linked to a trafficking sequence, wherein the DNA does not encode full-length CEA; and
(b) administering the fragment or DNA to the mammal.

61. A method of activating T cell responsiveness in a mammal, the method comprising:
(a) administering to the mammal (i) CEA or (ii) a DNA encoding CEA; and
(b) testing CD4⁺ T cells of the animal for responsiveness to a naturally processed fragment of CEA.

62. An isolated peptide comprising:
(a) at least one CEA segment selected from the group consisting of SEQ ID NO:1, SEQ ID NO:7, and SEQ ID NO:14; and
(b) one or more amino acids at the C-terminus of the CEA segment, at the N-terminus of the CEA segment, or at both the C-terminus and the N-terminus of the CEA segment,
wherein the peptide has an amino acid sequence that is not identical to that of full-length CEA.

63. A method of activating T cell reactivity in a mammal, the method comprising:
(a) providing (i) the peptide of claim 62, or (ii) a DNA encoding a polypeptide selected from the group consisting of (1) the peptide, (2) the peptide plus an amino terminal methionine residue, and (3) either (1) or (2) linked to a trafficking sequence, wherein the DNA does not encode full-length CEA; and
(b) administering the peptide or DNA to the mammal.
